(19) United States
(12) Patent Application Publication
(10) Pub. No.: US 2004/0170954 A1
(43) Pub. Date: Sep. 2, 2004

(54) Pathogen inactivation assay

(76) Inventors: Keith McKenney, Potomac, MD (US); Lidja Gillmeister, Fairfax, VA (US); Kristina Marlowe, Fairfax, VA (US); David Armistead, Arlington, VA (US)

Correspondence Address: FLESHNER & KIM, LLP, P.O. BOX 221200, CHANTILLY, VA, 20153, US

(21) Appl. No.: 10/361,002

(22) Filed: Feb. 10, 2003

(51) Int. Cl. C12Q 1/70; C12Q 1/68; C12P 19/34

(52) U.S. Cl. 435/5; 435/6; 435/91.2

(57) ABSTRACT

The present invention relates to methods for determining the level of potentially active biological pathogens, such as viruses, bacteria (including inter- and intracellular bacteria, such as mycoplasmas, ureaplasmas, nanobacteria, chlamydia, rickettsias), fungi (including yeasts) and single cell parasites, which may be found in a biological material. The present invention particularly relates to methods of determining the level of potentially active biological pathogens in a biological material using quantitative PCR.

### BACKGROUND OF THE INVENTION

1. Field of the Invention

The present invention relates to methods for determining the level of potentially active biological pathogens, such as viruses, bacteria (including inter- and intracellular bacteria, such as mycoplasmas, ureaplasmas, nanobacteria, chlamydia, rickettsias), fungi (including yeasts), and single- and multi-cell parasites, which may be found in a biological material. The present invention particularly relates to methods of determining the level of potentially active biological pathogens in a biological material using quantitative PCR, and so may be particularly useful for determining the effectiveness of a sterilization process that has been applied to the biological material.

2. Background of the Related Art

Many biological materials that are prepared for human, veterinary, diagnostic and/or experimental use may contain unwanted and potentially dangerous biological pathogens, such as viruses, bacteria, in both vegetative and spore states, (including inter- and intracellular bacteria, such as mycoplasmas, ureaplasmas, nanobacteria, chlamydia, rickettsias), fungi (including yeasts), and single- and multi-cell parasites. This may also be true of biological materials that are produced in or exported from locations where certain biological pathogens may exist to locations where those biological pathogens are not endemic. Consequently, it is of utmost importance that any biological pathogen in the biological material be inactivated before the material is used. This is especially critical when the biological material is to be administered directly to a patient, for example in tissue implants, blood transfusions, blood factor replacement therapy, organ transplants, and other forms of human and/or other animal therapy corrected or treated by surgical implantation, intravenous, intramuscular or other forms of injection or introduction. This is also critical for the various biological materials that are prepared in media or via the culture of cells, or recombinant cells which contain various types of plasma and/or plasma derivatives or other biological materials and which may be subject to mycoplasmal, prion, ureaplasmal, bacterial, viral and/or other biological pathogens.

All living cells and multi-cellular organisms can be infected with viruses and other pathogens. Thus, the products of unicellular natural or recombinant organisms or tissues virtually always carry a risk of pathogen contamination. In addition to the risk that the producing cells or cell cultures may be infected, the processing of these and other biological materials also creates opportunities for environmental contamination. The risks of infection are more apparent for multi-cellular natural and recombinant organisms, such as transgenic animals.

Interestingly, even products from species as different from humans as transgenic plants carry risks, both due to processing contamination as described above, and from environmental contamination in the growing facilities, which may be contaminated by pathogens from the environment or infected organisms that co-inhabit the facility along with the desired plants. For example, a crop of transgenic corn grown in a field could be expected to be exposed to rodents such as mice during the growing season. Mice can harbor serious human pathogens such as the frequently fatal Hanta virus. Since these animals would be undetectable in the growing crop, viruses shed by the animals could be carried into the transgenic material at harvest. Indeed, such rodents are notoriously difficult to control, and may gain access to a crop during sowing, growth, harvest or storage. Likewise, contamination from overflying or perching birds has the potential to transmit such serious pathogens as the causative agent for psittacosis. Thus, any biological material, regardless of its source, may harbor serious pathogens that must be removed or inactivated prior to administration of the material to a recipient human or other animal.

Accordingly, many procedures for producing human compatible biological materials have involved methods that screen or test the biological materials for one or more particular biological pathogens rather than removal or inactivation of the pathogen(s) from the biological material. The typical protocol for disposition of materials that test positive for a biological pathogen simply is non-use/discarding of that material.

Examples of screening procedures for contaminants include testing for a particular virus in human blood and tissues from donors. Such procedures, however, are not always reliable and are not able to detect the presence of certain viruses, and prions, particularly those present in very low numbers. This reduces the value, certainty, and safety of such tests in view of the consequences associated with a false negative result, which can be life threatening in certain cases, for example in the case of Acquired Immune Deficiency Syndrome (AIDS). Furthermore, in some instances it can take weeks, if not months, to determine whether or not the material is contaminated. Moreover, to date, there is no commercially available, reliable test or assay for identifying ureaplasmas, mycoplasmas, and chlamydia within a biological material that is fully suitable for screening out potential donors or infected material (Advances in Contraception 10(4):309-315(1994)). This serves to heighten the need for an effective means of destroying ureaplasmas, mycoplasmas, chlamydia, etc., within a biological material, while still retaining the desired activity of that material. Therefore, it is highly desirable to apply techniques that kill or inactivate biological pathogens during and/or after manufacturing and/or harvesting the biological material.

More recent efforts have focussed on methods to remove or inactivate contaminants in products intended for use in humans and other animals. Particularly useful methods are those that alter the genetic material of a biological pathogen, such as the addition of chemical inactivants or sensitizers to a biological material or irradiation of a biological material.

The use of chemical inactivants or sensitizers involves the addition of noxious agents which bind to the DNA/RNA of the virus, and which are activated either by UV or other radiation. This radiation produces reactive intermediates and/or free radicals which bind to the DNA/RNA of the virus, break the chemical bonds in the backbone of the DNA/RNA, and/or cross-link or complex it in such a way that the virus can no longer replicate.

Irradiating a biological material with ionizing radiation, such as gamma, UV or e-beam radiation, is another method of sterilizing a product. The direct effects of gamma radiation are particularly useful for destroying the genetic material within viruses and bacteria, particularly when given in total doses of at least 25 kGy (See Keathly, et al., “Is There Life After Irradiation? Part 2,” BioPharm July-August, 1993, and Leitman, “Use of Blood Cell Irradiation in the Prevention of Post Transfusion Graft-vs-Host Disease,” Transfusion Science 10:219-239(1989)).

The use of such sterilization methods does not, however, remove any biological pathogens from the sterilized biological material. Rather, these methods render inactive any biological pathogens that may be present in the biological material by altering the genetic material within the pathogen, including cleaving, deleting, oxidizing, reducing, covalently bonding, cross-linking and/or complexing that genetic material or a component thereof.

For many potentially active biological pathogens, a single modification in their genome may be sufficient to render them inactive. Significantly, most presently available tests for the detection or quantification of biological pathogens, such as ELISA tests for surface antigens, will not indicate that the biological pathogen has been rendered inactive. Moreover, conventional genetic detection tests, such as the PCR reaction described below, examine only a small portion of the genome and may fail to detect sites of alteration that render the biological pathogen inactive, irrespsective of whether there ate several such sites or only one. In practice, these tests may frequently report a high level of false positive results, leading to inappropriate product quarantine or destruction.

Accordingly, there is a need for methods to examine the genomes of potentially active biological pathogens in biological materials that have been subjected to sterilization in order to differentiate between biological pathogens that have been rendered inactive and those that are still potentially active. By doing so, such a method permits the determination of how effective a particular sterilization technique may be with respect to a particular biological pathogen. No presently available test provides such information.

PCR (polymerase chain reaction) is a method for increasing the concentration of a segment of a target sequence in a mixture of nucleic acid sequences without cloning or purification. (See K. B. Mullis et al., U.S. Pat. Nos. 4,683,195 and 4,683,202). This process for amplifying the target sequence consists of introducing two oligonucleotide primers to the sample containing the desired target nucleic acid sequence, followed by thermal cycling in the presence of a DNA polymerase. The two primers are complementary to their respective strands of the target sequence. To effect amplification, the genetic material within the sample is first denatured and then the primers are annealed to their complementary sequences within the target molecule. Following annealing, the primers are extended with a polymerase so as to form a new pair of complementary strands. The steps of denaturation, annealing and extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. Because the desired amplified segments of the target sequence become the predominant sequences (in terms of concentration) in the mixture, they are said to be “PCR amplified”. The segment of genetic material that has been amplified is generally referred to as an “amplicon”. The conditions employed for PCR reactions, including aspects of the timing, temperature(s) and particular polymerase selection are typically optimized for examining relatively short segments of nucleic acids, generally in the range of 50-200 nucleic acid residues. With PCR, it is possible to amplify a single copy of a specific target sequence in genomic DNA to a level detectable by several different methodologies (e.g., hybridization with a labelled probe; incorporation of biotinylated primers followed by avidin-enzyme conjugate detection; incorporation of ³²P-labelled deoxynucleotide triphosphates, e.g., dCTP or dATP, into the amplified segment). In addition to genomic DNA, any oligonucleotide sequence can be amplified with the appropriate set of primer molecules.

End-point PCR is a polynucleotide amplification protocol. The amplification factor that is observed is related to the number (n) of cycles that have occurred and the efficiency of replication at each cycle (E), which, in turn, is a function of the priming and extension efficiencies during each cycle. Amplification has been observed to follow the form Eⁿ, until high concentrations of the PCR product have been made.

At these high product concentrations, the efficiency of replication tends to drop significantly. It has been suggested that this is probably due to the displacement of the primers by the longer complementary strands of the PCR product. At concentrations in excess of 10⁻⁸ M, the rate of the two complementary PCR amplified product strands finding each other during the priming reactions becomes sufficiently fast that it may occur before or concomitantly with the extension step of the PCR process. This ultimately leads to a reduced priming efficiency, and, consequently, a reduced cycle efficiency. Continued cycles of PCR lead to declining increases of PCR product molecules, until the PCR product eventually reaches a plateau concentration (the “end-point”), usually a concentration of approximately 10⁻⁸ M. As a typical reaction volume is about 100 microlitets, this corresponds to a yield of about 6×10¹¹ double stranded product molecules.

Real-time PCR is also a polynucleotide amplification protocol, but PCR product analysis occurs simultaneously with amplification of the target sequence. Detecting agents, such as DNA dyes or fluorescent probes, can be added to the PCR mixture before amplification and used to analyze PCR products during amplification. Sample analysis occurs concurrently with amplification in the same tube within the same instrument. This combined approach decreases sample handling, saves time, and greatly reduces the risk of product contamination, as there is no need to remove the samples from their closed containers for further analysis. The concept of combining amplification with product analysis has become known as “real time” or “quantitative” PCR. (See, e.g., WO/9746707A2, WO/9746712A2 and WO/9746714A1).

Originally, monitoring fluorescence each cycle of PCR involved the use of ethidium bromide. See Higuchi et al., “Simultaneous amplification and detection of specific DNA sequences,” Bio/Technology 10:413-417 (1992); Higuchi et al., “Kinetic PCR analysis: real time monitoring of DNA amplification reactions,” Bio/Technology 11:1026-1030 (1993). In that system, fluorescence was measured once per cycle as a relative measure of product concentration. Ethidium bromide detects double stranded DNA; thus, if the desired target nucleic acid sequence is present, fluorescence intensity increases with temperature cycling (otherwise no fluorescence). Furthermore, the cycle number where an increase in fluorescence is first detected increases inversely proportionally to the log of the initial target sequence concentration. Other fluorescent systems have since been developed that are capable of providing additional data concerning the nucleic acid concentration.

In view of the difficulties and problems discussed above, there remains a need for a simple, yet accurate method of determining the efficiency of methods of sterilizing biological materials that act upon the genetic material of potentially active biological pathogens.

Each of the above references is incorporated by reference herein where appropriate for teachings of additional or alternative details, features and/or technical background.

### SUMMARY OF THE INVENTION

An object of the invention is to solve at least the problems and/or disadvantages of the relevant art, and to provide at least the advantages described hereinafter.

Accordingly, it is an object of the present invention to provide methods of determining the level of potentially active biological pathogens in a biological material. Other objects, features and advantages of the present invention will be set forth in the detailed description of preferred embodiments that follows, and in part will be apparent from the description or may be learned by practice of the invention. These objects and advantages of the invention will be realized and attained by the compositions and methods particularly pointed out in the written description and claims hereof.

In accordance with these and other objects, a first embodiment of the present invention is directed to a method for determining the level of potentially active biological pathogens in a biological material, which comprises: (i) adding to a biological material an effective amount of at least two nucleic acid primer pairs, wherein a first nucleic acid primer pair hybridizes under stringent conditions to a first target nucleic acid sequence found in the biological pathogen and a second nucleic acid primer pair hybridizes under stringent conditions to a second target nucleic acid sequence found in the biological pathogen, and further wherein first and second target nucleic acid sequences are not identical and the second target nucleic acid sequence contains more nucleic acid residues than the first; (ii) amplifying the target nucleic acid sequences by polymerase chain reaction, which comprises adding at least one polymerase to the biological material containing the primer pairs to form an amplification mixture and thermally cycling this amplification mixture between at least one denaturation temperature and at least one elongation temperature for a period of time sufficient to amplify the target nucleic acid sequences; and (iii) detecting and quantifying the target nucleic acid sequences, wherein the quantity of the first target nucleic acid sequence is proportional to the number of biological pathogens in the biological material and the quantity of the second target nucleic acid sequence is proportional to the number of potentially active biological pathogens in the biological material.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

FIG. 1 shows the complete genomic nucleic acid sequence of human Parvovirus B19, and indicates exemplary sequences for preparing suitable forward and reverse primers and probes.

FIG. 2 shows the complete genomic nucleic acid sequence of hepatitis B virus, and indicates exemplary sequences for preparing suitable forward and reverse primers and probes.

FIG. 3 shows the complete genomic nucleic acid sequence of porcine Parvovirus, and indicates exemplary sequences for preparing suitable forward and reverse primers and probes.

FIG. 4 shows the complete genomic nucleic acid sequence of Sindbis virus, and indicates exemplary sequences for preparing suitable forward and reverse primers and probes.

FIG. 5 shows the complete genomic nucleic acid sequence of West Nile virus, and indicates exemplary sequences for preparing suitable forward and reverse primers and probes.

FIGS. 6A and 6B show the genomic nucleic acid sequence of the 16S ribosomal RNA gene and the 23S ribosomal RNA gene of Escherichia coli, and indicate exemplary sequences for preparing suitable forward and reverse primers and probes.

FIGS. 7A and 7B show the genomic nucleic acid sequence of the 18S ribosomal RNA gene and the 25S ribosomal RNA gene of yeast (S. cerevisiae), and indicate exemplary sequences for preparing suitable forward and reverse primers and probes.

FIG. 8 shows the complete nucleic acid sequence of human mitochondrial DNA, and indicates exemplary sequences for preparing suitable forward and reverse primers and probes.

### DETAILED DESCRIPTION OF PREFERRED EMBODIMENTS

A. Definitions

Unless defined otherwise, all technical and scientific terms used herein are intended to have the same meaning as is commonly understood by one of ordinary skill in the relevant art.

As used herein, the singular forms “a,” “an,” and “the” include the plural reference unless the context clearly dictates otherwise.

As used herein, the term “biological material” is intended to mean any substance derived or obtained from a living organism. Illustrative examples of biological materials include, but are not limited to, the following: cells; tissues; blood or blood components; proteins, including recombinant and transgenic proteins, and proetinaceous materials; enzymes, including digestive enzymes, such as trypsin, chymotrypsin, alpha-galactosidase and iduronodate-2-sulfatase; immunoglobulins, including mono and polyimmunoglobulins; botanicals; food and the like. Preferred examples of biological materials include, but are not limited to, the following: ligaments; tendons; nerves; bone, including demineralized bone matrix, grafts, joints, femurs, femoral heads, etc.; teeth; skin grafts; bone marrow, including bone marrow cell suspensions, whole or processed; heart valves; cartilage; corneas; arteries and veins; organs, including organs for transplantation, such as hearts, livers, lungs, kidneys, intestines, pancreas, limbs and digits; lipids; carbohydrates; collagen, including native, afibrillar, atelomeric, soluble and insoluble, recombinant and transgenic, both native sequence and modified; chitin and its derivatives, including NO-carboxy chitosan (NOCC); stem cells, islet of Langerhans cells and other cells for transplantation, including genetically altered cells; red blood cells; white blood cells, including monocytes; and platelets. Additional examples of biological materials include forensic samples, human or animal remains, stomach contents, mummified remains of a once-living organism, fossilized remains, a product of manufacture containing or previously in contact with a biological material, and fomites.

As used herein, the term “biological pathogen” is intended to mean a biological pathogen that, upon direct or indirect contact with a biological material, may have a deleterious effect on the biological material or upon a recipient thereof. Such biological pathogens include, but are not limited to, the various viruses, bacteria (whether in the vegetative or spore state, including inter- and intracellular bacteria, such as mycoplasmas, ureaplasmas, nanobacteria, chlamydia, rickettsias), fungi (including yeasts) and/or single- or multi-cell parasites and pests known to those of skill in the art to generally be found in or infect biological materials.

Illustrative examples of some biological pathogens include, but are not limited to, the following: viruses, such as human immunodeficiency viruses and other retroviruses, herpes viruses, filoviruses, circoviruses, paramyxoviruses, cytomegaloviruses, hepatitis viruses (including hepatitis A, B, C, and D variants thereof, among others), pox viruses, toga viruses, Ebstein-Barr viruses and parvoviruses; bacteria, such as Escherichia, Bacillus, Campylobacter, Clostridium, Streptococcus and Staphylococcus; nanobacteria; single- and multi-cell parasites, such as Trypanosoma and malarial parasites, including Plasmodium species; fungi; yeasts; mycoplasmas and ureaplasmas; chlamydia; rickettsias, such as Coxiella burnetti; and multi-cell pests and the like.

Additional non-limiting examples of pathogens found in biological materials include the following bacteria: Escherichia, Bacillus, Campylobacter, Helicobacter, Lysteria, Clostridium, Streptococcus, Enterococcus, Staphylococcus, Brucella, Haemophilus, Salmonella, Yersinia, Pseudomonas, Serratia, Enterobacter, Kebsiella, Proteus, Citrobacter, Corynebacterium, Propionibacterium and Coxiella, such as Staphylococci (including, for example, S. aureus, S. epidermidis, S. saprophyticus, among others), Chlamydia (including, for example, C. pneumoniae, among others), Streptococci (including, for example, the viridians group of Streptococci: S. sanguis, S. oralis (mitis), S. salivatius, S. mutans, and others; and other species of Streptococci, such as S. bovis and S. pyogenes), Enterococci (for example, E. faecalis and E. faecium, among others), various fungi, and the AHACEK group of gram-negative bacilli (Haemophilus parainfluenzae, Haemophilus aphrophilus, Actinibacillus actnomycetemcomitans, Cardiobacterium hominis, Eikenella corrodens, and Kingella kingae), Neisseria gonorrhoeae, Clostridia sp., Listeria moncytogenes, Salmonella sp., Bacteroides fragilis, Escherichia coli, Proteus sp, and Klebsiella-Enterobacter-Serratia sp., among others.

Still other non-limiting examples of pathogens found in biological materials include the following viruses: Adeno-associated Virus (AAV), California Encephalitis Virus, Coronavirus, Coxsackievirus-A, Coxsackievirus-B, Eastern Equine Encephalitis Virus (EEEV), Echovirus, Hantavirus, Hepatitis A Virus (HAV), Hepatitis C Virus (HCV), Hepatitis Delta Virus (HDV), Hepatitis E Virus (HEV), Hepatitis G Virus (HGV), Human Immunodeficiency Virus (HIV), Human T-lymphotrophic Virus (HTLV), Influenza Virus (Flu Virus), Measles Virus (Rubeola), Mumps Virus, Norwalk Virus, Parainfluenza Virus, Polio virus, Rabies Virus, Respiratory Syncytial Virus, Rhinovirus, Rubella Virus, Saint Louis Encephalitis Virus, Western Equine Encephalitis Virus (WEEV), Yellow Fever Virus, Adenovirus, Cytomegalovirus (CMV), Epstein-Barr Virus (EBV), Hepatitis B Virus (HBV), Herpes Simplex Virus 1 (HHV1), Herpes Simplex Virus 2 (HHV2), Molluscum contagiosum, Papilloma Virus (HPV), Smallpox Virus (Variola), Vaccinia Virus, Venezuelan Equine Encephalitis Virus (VEEV), Ebola Virus, West Nile Virus, Human Parvovirus B19 and Rotavirus.

As used herein, the term “potentially active biological pathogen” is intended to mean a biological pathogen that is capable of causing a deleterious effect, either alone or in combination with another factor, such as a second biological contaminant or pathogen or a native protein (wild-type or mutant) or antibody, in the biological material and/or a recipient thereof.

As used herein, the term “wild-type” in reference to a nucleic acid sequence an amino acid sequence is intended to refer to the corresponding sequence found in naturally occurring organisms, such as biological pathogens, including such variants and mutants as are known to those skilled in the art.

As used herein, the term “sterilize” is intended to mean a reduction in the level of at least one potentially active biological pathogen found in the biological material being treated.

As used herein, the term “radiation” is intended to mean radiation of sufficient energy to sterilize at least some component of the irradiated biological material. Types of radiation include, but are not limited to, the following: (i) corpuscular (streams of subatomic particles such as neutrons, electrons, and/or protons); (ii) electromagnetic (originating in a varying electromagnetic field, such as radio waves, visible (both mono and polychromatic) and invisible light, infrared, ultraviolet radiation, x-radiation, and gamma rays and mixtures thereof); and (iii) sound and pressure waves. Such radiation is often described as either ionizing (capable of producing ions in irradiated materials) radiation, such as gamma rays, and non-ionizing radiation, such as visible light. The sources of such radiation may vary and, in general, the selection of a specific source of radiation is not critical provided that sufficient radiation is given in an appropriate time and at an appropriate rate to effect sterilization. In practice, gamma radiation is usually produced by isotopes of Cobalt or Cesium, while UV and X-rays are produced by machines that emit UV and X-radiation, respectively, and electrons are often used to sterilize materials in a method known as “E-beam” irradiation that involves their production via a machine. Visible light, both mono- and polychromatic, is produced by machines and may, in practice, be combined with invisible light, such as infrared and UV, that is produced by the same machine or a different machine.

B. Particularly Preferred Embodiments

A first preferred embodiment of the present invention is directed to a method for determining the level of potentially active biological pathogens in a biological material, which comprises:

(i) adding to a biological material an effective amount of at least two nucleic acid primer pairs,

wherein a first nucleic acid primer pair hybridizes under stringent conditions to a first target nucleic acid sequence found in the biological pathogen and a second nucleic acid primer pair hybridizes under stringent conditions to a second target nucleic acid sequence found in the biological pathogen, and further wherein first and second target nucleic acid sequences are not identical and the second target nucleic acid sequence contains more nucleic acid residues than the first;

(ii) amplifying the target nucleic acid sequences by polymerase chain reaction, which comprises adding at least one polymerase to the biological material containing the primer pairs to form an amplification mixture and thermally cycling this amplification mixture between at least one denaturation temperature and at least one elongation temperature for a period of time sufficient to amplify the target nucleic acid sequences; and

(iii) detecting and quantifying the target nucleic acid sequences, wherein the quantity of the first target nucleic acid sequence is proportional to the number of biological pathogens in the biological material and the quantity of the second target nucleic acid sequence is proportional to the number of potentially active biological pathogens in the biological material.

The first and second target nucleic acid sequences employed in the methods of the present invention are preferably selected to be specific for a particular biological pathogen of interest. That is, it is preferred that at least one, and more preferably both, of the first and second nucleic acid sequences is found only in the biological pathogen of interest and not in any other component of the biological material. According to these embodiments of the present invention, such a selection (or selections) for the target nucleic acid sequence(s) allows for the selective determination of the levels of biological pathogen, including the total number of biological pathogens present (potentially active and inactive) as well as the number of potentially active pathogens and the number of inactive pathogens.

One skilled in the art may determine suitable target nucleic acid sequences empirically, based on factors such as the particular biological pathogen(s) of interest, the biological material being tested and the PCR conditions selected.

Preferably, the first target nucleic acid sequence contains between about 50 and about 500 nucleic acid residues. More preferably, the first target nucleic acid sequence contains between about 50 and about 250 nucleic acid residues, and most preferably between about 50 and about 150 nucleic acid residues.

The second target nucleic acid sequence preferably contains between about 500 and about 50,000 nucleic acid residues. More preferably, the second target nucleic acid sequence contains between about 1000 and about 10,000 nucleic acid residues, even more preferably between about 2000 and about 5000 nucleic acid residues and most preferably between about 2500 and about 5000 nucleic acid residues.

The first and second target nucleic acid sequences may be completely different or they may overlap by some or all of the shorter of the two. According to certain preferred embodiments of the present invention, the first target nucleic acid sequence and the second nucleic acid sequence contain at least 16 contiguous nucleic acid residues in common.

As noted, the first and second target nucleic acid sequences are preferably selected to be specific for a biological pathogen of interest. According to such embodiments of the present invention, the biological pathogen is preferably selected from the group consisting of bacteria, viruses, mycoplasmas, fungi and single cell parasites.

According to these embodiments of the present invention, at least one of the first and second target nucleic acid sequences, and more preferably both the first and second target nucleic acid sequence, are at least 30% homologous to a wild-type nucleic acid sequence found in the biological pathogen of interest. More preferably, the first and/or second target nucleic acid sequence is at least 50% homologous to a wild-type nucleic acid sequence found in the biological pathogen of interest, and even more preferably at least 70% homologous. Most preferably, the first and/or second target nucleic acid sequence is at least 90% homologous to a wild-type nucleic acid sequence found in the biological pathogen of interest. According to certain preferred embodiments, the first and/or second target nucleic acid sequence is substantially identical to a wild-type nucleic acid sequence found in the biological pathogen of interest.

According to particularly preferred embodiments of the present invention, at least one of the first and second target nucleic acid sequences, and more preferably both, is a nucleic sequence that is highly conserved among different species, different genera or even different families of biological pathogens.

For example, if the biological pathogen of interest is bacteria, then the first and/or second target nucleic acid sequences are preferably sequences that are found in the gene encoding the 16S ribosomal RNA or the gene encoding the 23S ribosomal RNA. According to these preferred embodiments of the present invention, the first target nucleic acid sequence is even more preferably a nucleic acid sequence found in the gene encoding the 16S ribosomal RNA of bacteria. Preferably, such a sequence is conserved among different species and genera of bacteria.

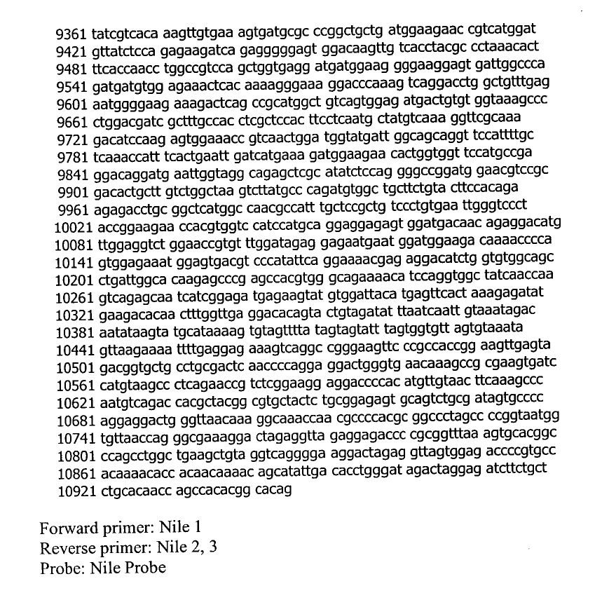

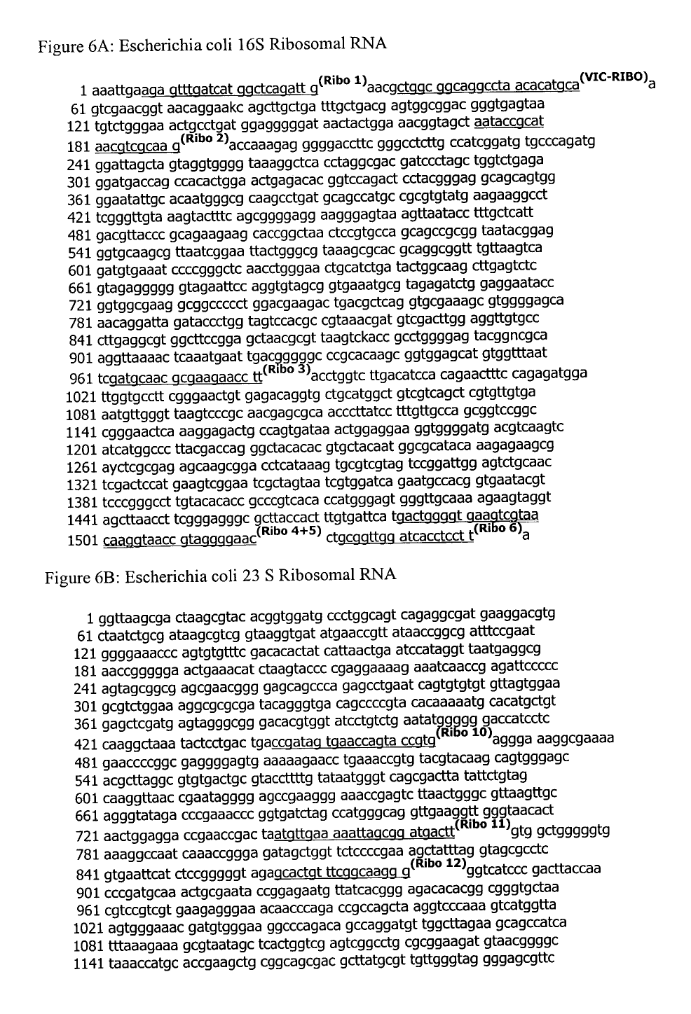

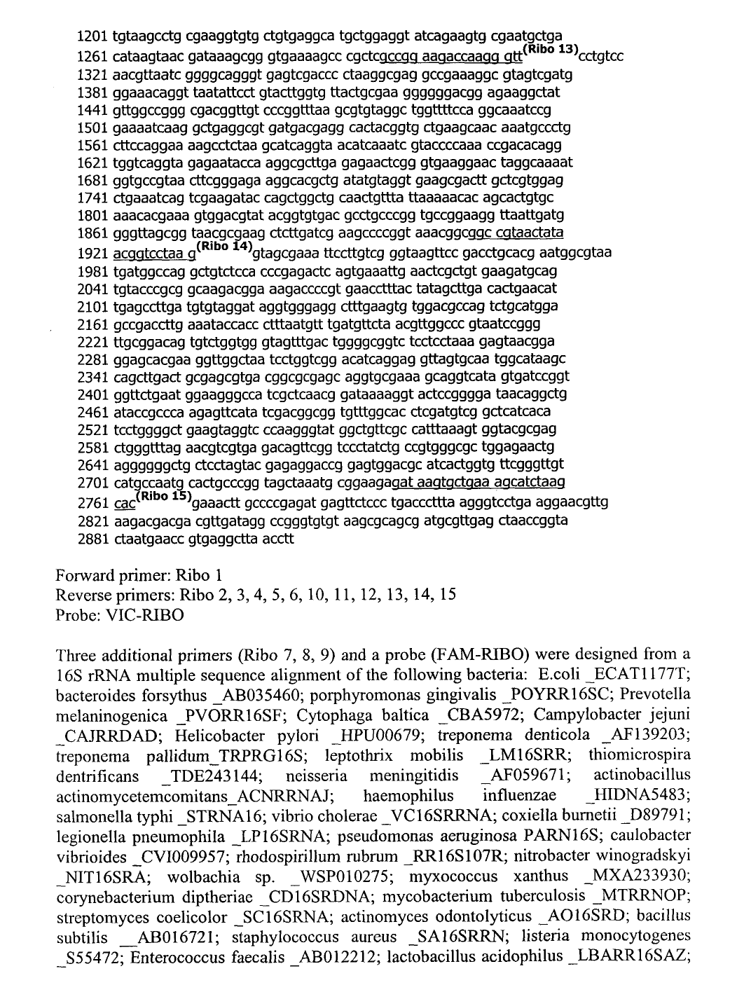

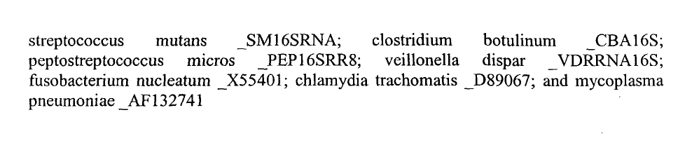

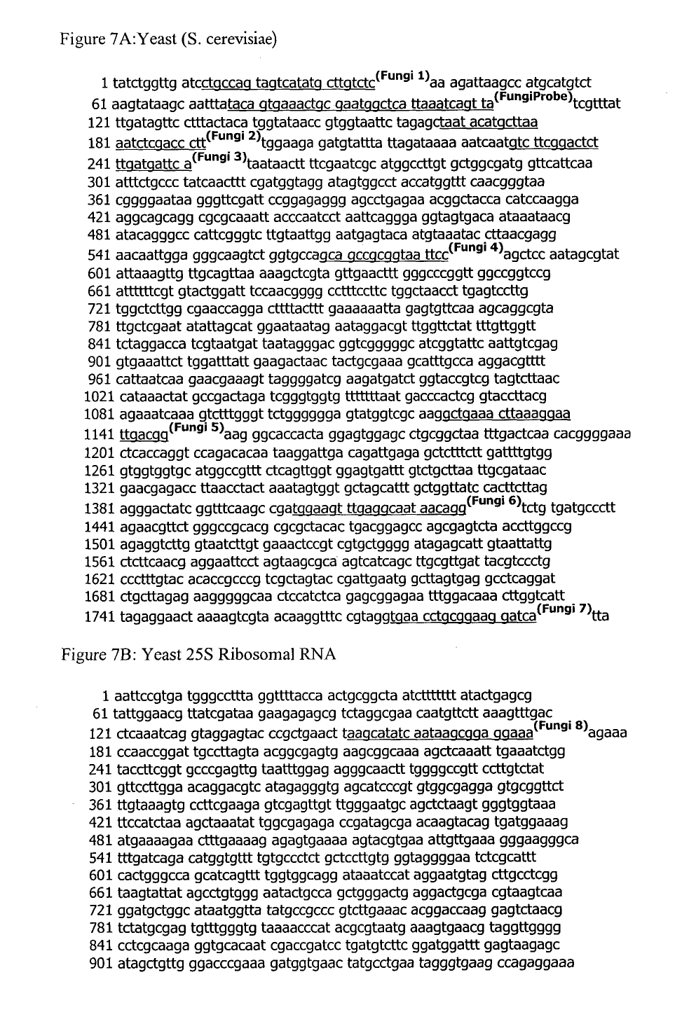

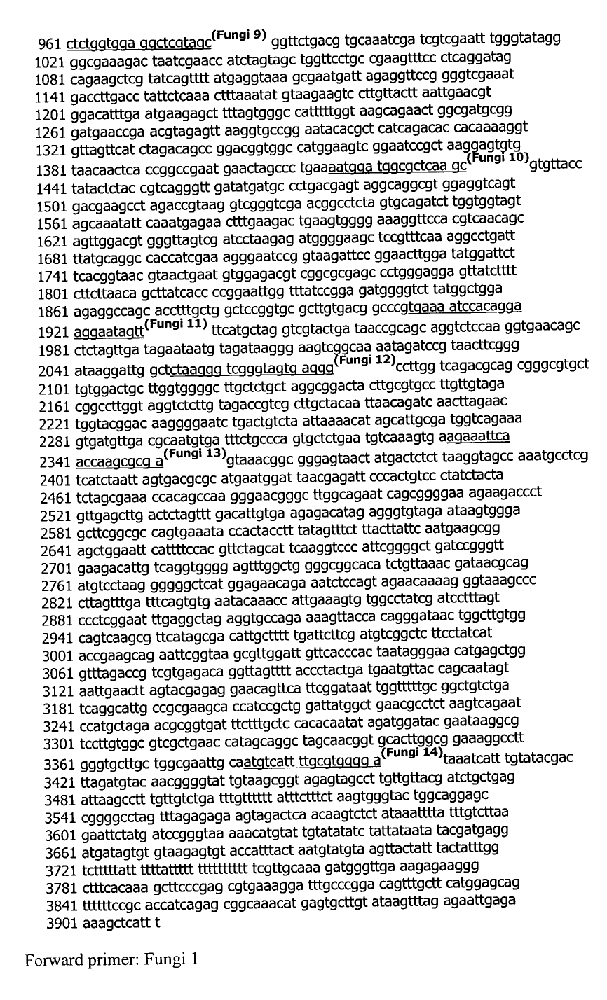

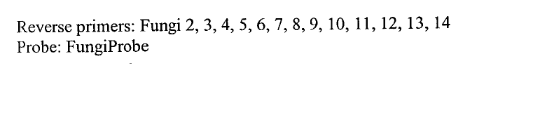

Thus, as shown in FIG. 6A, suitable primers and probes were prepared from the gene encoding the 16S ribosomal RNA of bacteria that were useful for a wide range of bacterial biological pathogens, including Escerichia coli, Bacteroides forsythus, Porphyromonas gingivalis, Prevotella melaninogenica, Cytophaga baltica, Campylobacter jejuni, Helicobacter pylori, Trepnema denticola, Treponema pallidum, Leptothrix mobilis, Thiomicrospira dentrificans, Neisseria meningitides, Actinobacillus actinomycetemcomitans, Haemophilus influenzae, Salmonella typhi, Vibrio cholerae, Coxiella burnettti, Legionella pneumophila, Pseudomonas aeruginosa, Caulobacter vibrioides, Rhodospirillum rubrum, Nitrobacter winogradskyi, Wolbachia sp., Myxococcus xanthus, Corynebacterium diptheriae, Mycobacterium tuberculosis, Streptomyces coelicolor, Actinomyces odontolyticus, Bacillus subtilis, Staphylococcus aureus, Listeria monocytogenes, Enterococcus faecais, Lactobacillus acidophilus, Streptococcus mutans, Clostridium botulinum, Peptostreptococcus micros, Veillonella dispar, Fusobacterium nucleatum, Clamydia trachomatis, Mycoplasma pneumoniae.

According to these particularly preferred embodiments of the present invention, i.e. if the biological pathogen of interest is bacteria, then the second target nucleic acid sequence is preferably a nucleic acid sequence found in both the gene encoding the 16S ribosomal RNA and the gene encoding the 23S ribosomal RNA. According to these embodiments, the second target nucleic acid sequence is even more preferably a nucleic acid sequence found in the gene encoding the 16S ribosomal RNA and at least a portion of the gene encoding the 23S ribosomal RNA, as well as the non-coding nucleic sequence found therebetween in bacterial genomes.

Similarly, if the biological pathogen of interest is fungi, then the first and/or second target nucleic acid sequences are preferably sequences that are found in the gene encoding the 18S ribosomal RNA or the gene encoding the 25S ribosomal RNA. According to these embodiments of the present invention, the first target nucleic acid sequence is even more preferably a nucleic acid sequence found in the gene encoding the 18S ribosomal RNA of fungi. Preferably, such a sequence is conserved among different species and genera of fungi.

According to these particularly preferred embodiments of the present invention, i.e. if the biological pathogen of interest is fungi, then the second target nucleic acid sequence is preferably a nucleic acid sequence found in both the gene encoding the 18S ribosomal RNA and the gene encoding the 25S ribosomal RNA. According to these embodiments, the second target nucleic acid sequence is even more preferably a nucleic acid sequence found in the gene encoding the 18S ribosomal RNA and at least a portion of the gene encoding the 25S ribosomal RNA and the non-coding nucleic sequence found therebetween in fungal genomes, and most preferably a nucleic acid sequence found in the gene encoding the 18S ribosomal RNA, at least a portion of the gene encoding the 25S ribosomal RNA and the gene encoding the 5.8S ribosomal RNA, as well as both non-coding nucleic sequences found therebetween in fungal genomes.

The first and second pairs of nucleic acid primers are each selected based on their ability to generate the desired target nucleic acid sequences under the appropriate PCR conditions. Accordingly, each primer must be specific for the desired target nucleic acid sequence. Similarly, each primer must be selected so that they are not self-complementary or complementary to another primer (or probe, if present).

According to certain preferred embodiments of the present invention, at least one member of each pair of nucleic acid primers is substantially identical, i.e. one of the first pair of nucleic acid primers and one of the second pair of nucleic acid primers are substantially identical.

According to other preferred embodiments of the present invention, the two pairs of nucleic acid primers are completely different, i.e., neither of the first pair of nucleic acid primers is substantially identical to either of the second pair of nucleic acid primers.

According to still other preferred embodiments of the present invention, the two pairs of nucleic acid primers are substantially identical, i.e. one of the first pair of nucleic acid primers is substantially identical to one of the second pair of nucleic acid primers and the other one of the first pair is identical to the other one of the second pair. According to such embodiments, two distinct target sequences may still be obtained, for example, in the case where one or both members of each primer pair hybridize to more than one sequence, for example, as in the case where the first and second target sequences ate part of a circular nucleic acid sequence, such as a plasmid, where the hybridization location of the primers on the circular nucleic acid sequence is such that transcription in different directions leads to two different amplicons. Similarly, in cases where the first and target sequences are highly homologous, particularly at their respective 5′ and 3′ ends, then the primers will hybridize to both, such that transcription leads to two different amplicons.

The polymerize chain reaction employed in the inventive methods is performed according to the methods and techniques known to those skilled in the art, i.e., a nucleic acid primer pair is added to the biological material containing the sequence of interest to form an amplification mixture that is then thermally cycled for a sufficient period of time to amplify the desired sequence. The thermal cycling generally comprises cycling the amplification mixture between at least one denaturation temperature and at least one elongation temperature. Preferably, the thermal cycling comprises cycling the amplification mixture between at least one denaturation temperature, at least one annealing temperature and at least one elongation temperature.

Specific temperatures for use in denaturation, elongation and/or annealing may be determined empirically by one skilled in the art based, for example, on the specific target sequence being amplified and the particular probes employed. Likewise, the specific time(s) that the amplification mixture is maintained at the various denaturation, elongation and/or annealing temperature(s) may be determined empirically by one skilled in the art based on similar considerations.

According to particularly preferred embodiments of the present invention, the elongation temperature selected for use in the PCR of the inventive methods is not more than about 70° C. More preferably, the elongation temperature selected is between about 60° C. and about 69° C., and even more preferably between about 65° C. and about 69° C. Most preferably, the elongation temperature employed in the PCR of the inventive methods is about 68° C.

According to additional preferred embodiments of the present invention, the denaturation temperature selected for use in the PCR of the inventive methods is not more than about 95° C. More preferably, the denaturation temperature selected is between about 90° C. and about 95° C., and even more preferably between about 92° C. and about 95° C. Most preferably, the denaturation temperature employed in the PCR of the inventive methods is about 94° C.

According to other preferred embodiments of the present invention, when the thermal cycling includes an annealing temperature, the annealing temperature selected is about 5-10° C. below the melting temperature of the primers being employed. Preferably, the annealing temperature selected is not more than about 65° C. More preferably, the annealing temperature selected is between about 57° C. and about 63° C., and even more preferably between about 58° C. and about 62° C. Most preferably, the annealing temperature employed in the PCR of the inventive methods is about 60° C.

According to additional preferred embodiments of the present invention, during each thermal cycle, the amplification mixture is maintained at the elongation temperature for a period of not less than about 1 minute. More preferably, during each thermal cycle, the amplification mixture is maintained at the elongation temperature for a period of not less than about 2 minutes, and even more preferably for a period of not less than about 3 minutes.

According to particularly preferred embodiments of the present invention, the amplification mixture is maintained at the elongation temperature for a period of not less than about 2 minutes during the first cycle of the thermal cycling, and then the period during which said amplification mixture is maintained at the elongation temperature is increased by a period of about 5 seconds for each successive thermal cycle. Thus, for example, according to such embodiments of the present invention, if the amplification mixture was maintained at the elongation temperature for a period of 2 minutes during the first cycle of the thermal cycling, it would be maintained at the elongation temperature for a period of 2 minutes, 5 seconds for the second cycle, 2 minutes, 10 seconds for the third cycle, 2 minutes, 15 seconds for the fourth cycle, and so on until the thermal cycling is completed.

According to additional preferred embodiments of the present invention, during each thermal cycle, the amplification mixture is maintained at the denaturation temperature for a period of not more than about 1 minute. More preferably, during each thermal cycle, the amplification mixture is maintained at the denaturation temperature for a period of not more than about 45 seconds, and even more preferably for a period of not more than about 30 seconds, and still even more preferably for a period of not more than about 20 seconds. Most preferably, during each thermal cycle, the amplification mixture is maintained at the denaturation temperature for a period of not more than about 15 seconds, such as a period of about 10 seconds.

According to still other preferred embodiments of the present invention, when the thermal cycling includes an annealing temperature, the amplification mixture is maintained at the annealing temperature for a period of not less than about 30 seconds. More preferably, according to such embodiments, during each thermal cycle, the amplification mixture is maintained at the annealing temperature for a period between 30 seconds and 2 minutes, and even more preferably for a period of not less than about 45 seconds. Most preferably, during each thermal cycle, the amplification mixture is maintained at the annealing temperature for a period of about 1 minute.

The number of thermal cycles employed in the PCR of the inventive methods may be determined empirically by one skilled in the art depending, for example, on the suspected concentration of the target sequence of interest in the biological material being tested. According to preferred embodiments of the present invention, the amplification mixture is subjected to at least about 30 cycles of thermal cycling, and even more preferably at least about 40 cycles. Most preferably, the amplification mixture is subjected to at least about 50 cycles of thermal cycling.

The polymerase employed in the PCR of the inventive methods may be any of the suitable polymerases known to those skilled in the art. Preferably, the polymerase employed is a thermostable polymerase, i.e. a polymerase that is not adversely affected by the higher temperatures involved in thermal cycling. More preferably, the polymerase may be a Taq polymerase, or a suitable derivative thereof and/or a proof-reading polymerase.

According to particularly preferred embodiments of the present invention, at least two polymerases are employed in the PCR of the inventive methods. Preferably, at least one of the polymerases is a Taq polymerase or a suitable derivative thereof, such as TaqMan DNA polymerase (available from Applied BioSystems), and the other polymerase is a proof-reading polymerase, such as ProofStart DNA polymerase (available from Qiagen).

According to certain preferred embodiments of the present invention, the amplification mixture further contains at least one thermostable inorganic pyrophosphatase. Suitable amounts of thermostable inorganic pyrophosphatase may be determined empirically by one skilled in art. Generally, when present, the ratio of thermostable inorganic pyrophosphatase to Taq polymerase is at least about 1:20, more preferably at least about 1:10 and even more preferably at least about 1:5.

The remaining parameters employed in the PCR of the inventive methods, such as the primer concentration (generally about 100-500 nM and preferably about 200 nM)), magnesium concentration (generally 1.5-6 mM and preferably about 1.5 mM of magnesium sulfate and/or magnesium chloride), deoxyribonucleotide triphosphates (dNTP) concentration (generally about 0.2-0.4 mM each and preferably about 0.2 mM each), probe concentration (if present, generally about 50-800 nM, and preferably about 100 nM), may each be determined empirically by one skilled in the art using any of the known methods and techniques.

According to certain particularly preferred embodiments of the present invention, the deoxyribonucleotide ttiphosphates (dNTP) that are employed in the PCR of the inventive methods are selected from the group consisting of C, T, G and A. Preferably, substantially no dUTP is present in the amplification mixture of the inventive methods. According to still further preferred embodiments, substantially no uracil N-glycosylase is present in the amplification mixture of the inventive methods.

According to certain particularly preferred embodiments of the present invention, the amplification mixture further comprises at least one buffer solution. Suitable buffer solutions are known and available to those skilled in the art. Particularly preferred buffer solutions include pH modifying buffers, such as buffers containing Tris-HCl, and buffers which maintain salt concentration, particular magnesium concentration, such as buffers containing KCl and/or (NH₄)₂SO₄.

After amplification using PCR, the first and second target nucleic acid sequences are detected and quantified. This detecting and quantifying may be conducted using any of the methods and techniques known to those skilled in the art. For example, detecting and quantifying of the first and second nucleic acid sequences may be conducted by adding a suitable detecting agent, such as an intercalating dye, directly to the amplification mixture or by adding a suitable nucleic acid probe to the mixture, preferably either a suitable nucleic acid probe in combination with a detecting agent or a suitable nucleic acid probe having a detectable label covalently or ionically attached thereto or complexed therewith.

Preferably, the first and second target nucleic acid sequences are detected by adding at least one nucleic acid probe to the biological material being tested. If the first and second target nucleic acid sequences were amplified in a single reaction vessel, then it is preferable to use at least two nucleic acid probes, one of which is specific for the first target nucleic acid sequence and the other of which is specific for the second target nucleic acid sequence. Conversely, if the first and second target nucleic acid sequences were amplified in separate reaction vessels, then the same nucleic acid probe may be used for detecting both the first target nucleic acid sequence and the second target nucleic acid sequence.

Any nucleic acid probe employed in the inventive methods should contain sufficient nucleic acid residues to hybridizes selectively under stringent conditions to a specific desired nucleic acid sequence, i.e. suitable probes will generally contain at least 16 nucleic acid residues, and preferably hybridizes selectively under stringent conditions to a specific nucleic acid sequence of the first and/or second target nucleic acid sequence that is not the same as the nucleic acid sequence of any of the primers. Suitable nucleic acid probes include, but are not limited to, 5′ nuclease probes, hairpin probes, adjacent probes, sunrise probes and scorpion probes.

According to certain preferred embodiments of the present invention, the nucleic acid probe employed in the inventive methods has an endogneous passive dye, such as Tamra or the like. In other preferred embodiments, such an endogenous passive dye may be replaced by a passive dye that is not covalently bound to the probe, such as Rox or the like.

According to certain particularly preferred embodiments of the present invention, prior to step (i), the biological material being tested has been subjected to a process that alters at least one wild-type nucleic acid sequence in the biological pathogen of interest. Such processes may cause the wild-type nucleic acid sequence to break, cross-link and/or complex. An illustrative, but non-limiting, example of such a process is irradiation of the biological material with ionizing radiation, such as UV or gamma radiation.

Althogh not limited in application, the inventive methods are particularly useful in determining the effectiveness of processes that alter nucleic acid sequences, such as the inactivation of biological pathogens by gamma irradiation. More specifically, conventional PCR testing methods only determine whether a particular biological pathogen is present in a biological material, not whether that biological pathogen is active or inactive. The methods of the present invention, however, may be used to determine not only whether a particular biological pathogen is present in a biological material as shown by amplification of the first target sequence, but also whether that biological pathogen is inactive by virtue of an altered wild-type nucleic acid sequence as shown by a relative delay in the amplification of the second target sequence (the greater the delay in amplification, the greater the reduction in the level of potentially active biological pathogens). Thus, the inventive methods are useful for evaluating the effectiveness of sterilization processes because they determine both the original level and the residual level of potentially active biological pathogens.

### EXAMPLES

The following examples are illustrative, but not limiting, of the present invention. Other suitable modifications and adaptations are of the variety normally encountered by those skilled in the art and are fully within the spirit and scope of the present invention.

### Example 1

Purpose: To demonstrate linear amplification of B19 DNA.

Materials: 1. B19 virus, titer 7.6×10¹¹ iu/ml from Bayer;

2. SNAP whole blood DNA isolation kit;

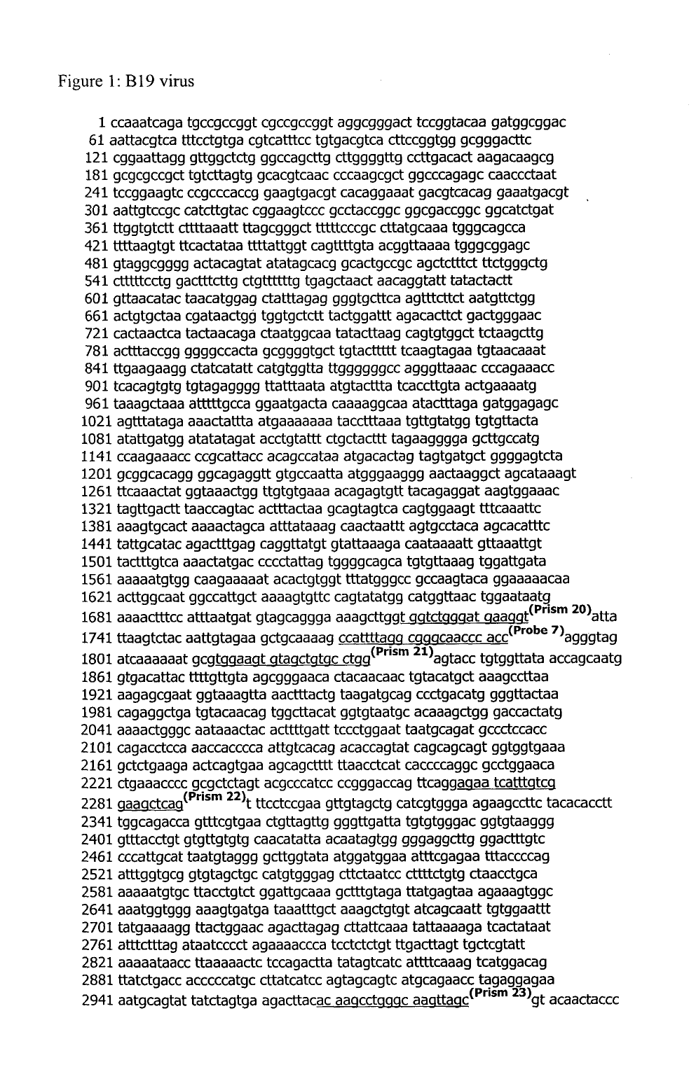

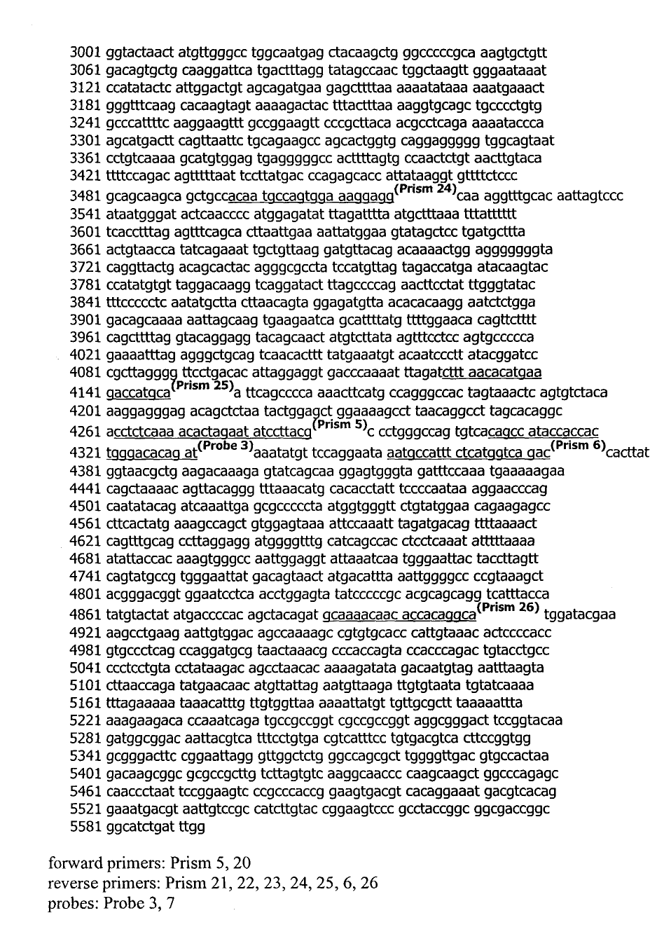

3. Forward Primer: Prism 5 (FIG. 1);

4. Reverse Primer: Prism 6 (FIG. 1);

5. Probe 3 (FIG. 1) labeled with FAM at 5′ end and TAMRA at 3′ end;

6. TaqMan Universal Master Mix, (ABI; cat. no. 4304437);

7. DNASE, RNASE free water;

8. ABI 96 well plate and adhesive cores;

9. ANI 7000.

Procedure: 1. Followed SNAP protocol for extraction of 100 μL B19 sample, eluted in 100 μl TE;

2. Diluted primers to 18 μM with TE;

3. Diluted probe to 5 μM with TE;

4. Prepared the following master mix:

| | |
|---|---|
| TaqMan Master Mix: | 25 μl; |
| Prism 5: | 2.5 μl; |
| Prism 6: | 2.5 μl; |
| Taqman Probe | 2.5 μl; |
| Water: | 12.54 μl; |

5. Added 45 μl of master mix per well;

6. Serially diluted B19 DNA, adding water to the NTC well;

7. Sealed and centrifuged the plate at 2300 rpm for about 30 seconds;

8. Ran PCR program for 50 cycles.

Results: A standard dilution curve was observed for B19 infected plasma, validating primer pair Prism 5 and Prism 6 with Probe 3.

### Example 2

Purpose: To examine irradiated and unirradiated samples containing PPV using a 549 bp amplicon.

Materials: 1. PPV (irradiated at 0 kGy, 50 kGy, 65 kGy, 75 kGy or 85 kGy);

2. SNAP Protein Degrader;

3. Cell Lysis Buffer;

4. Tris-HCl;

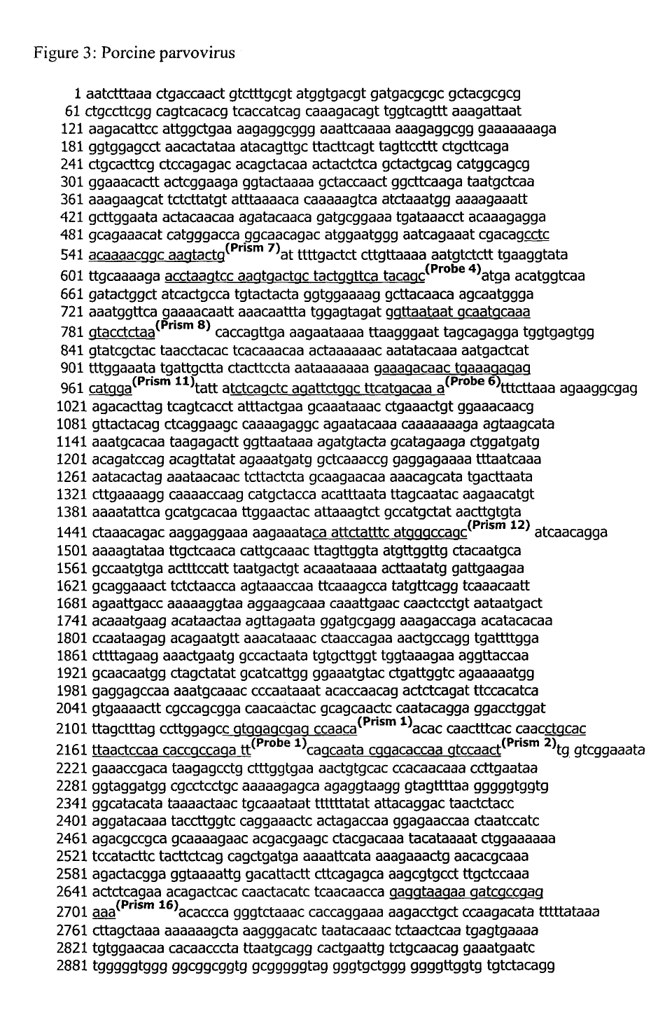

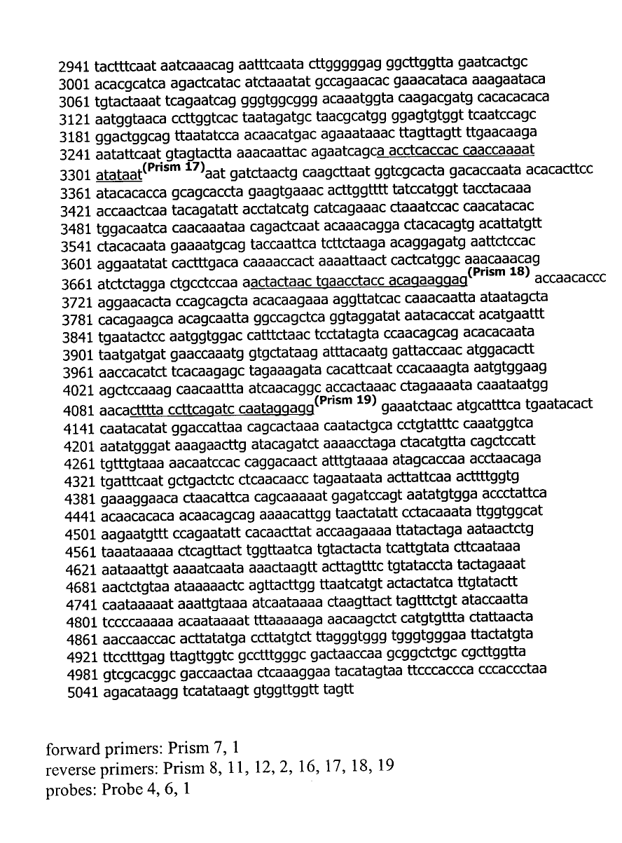

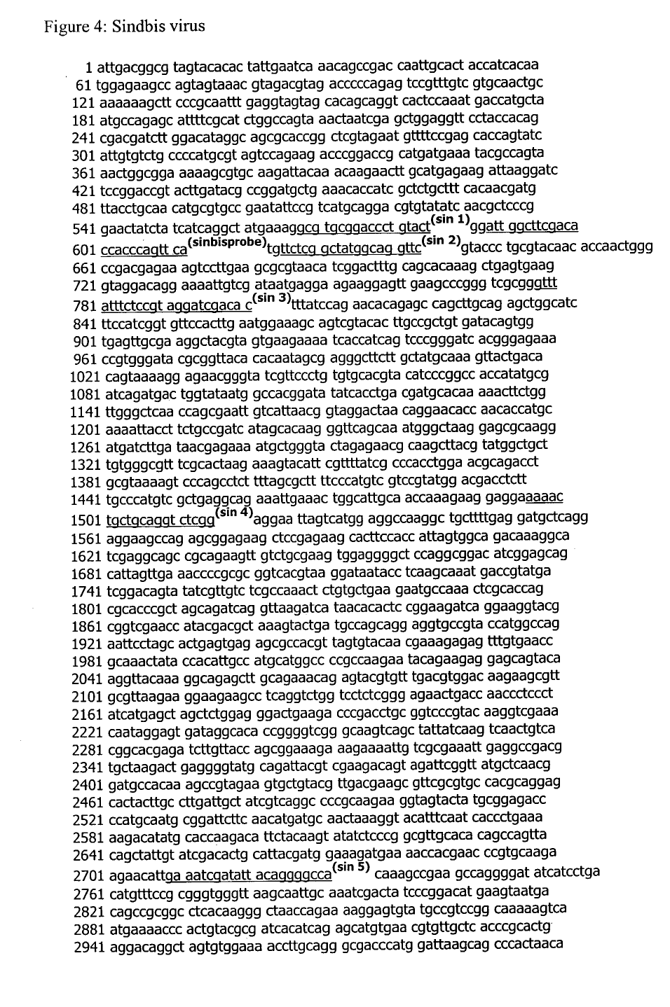

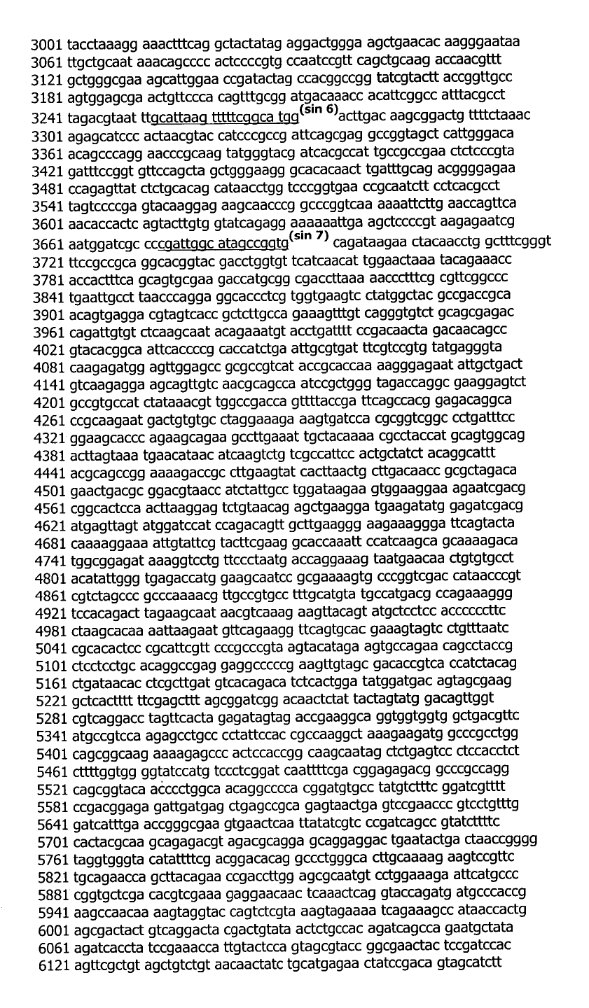

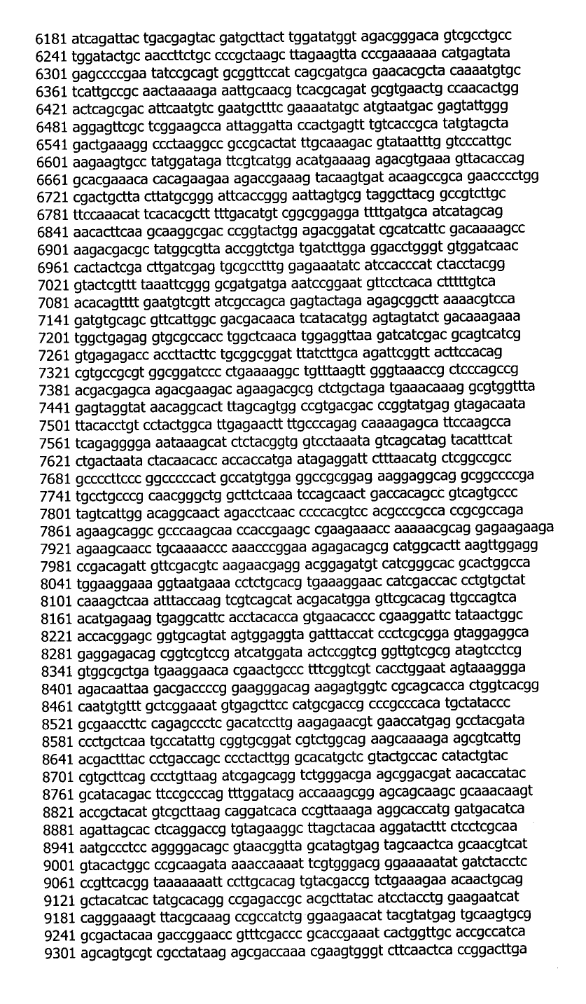

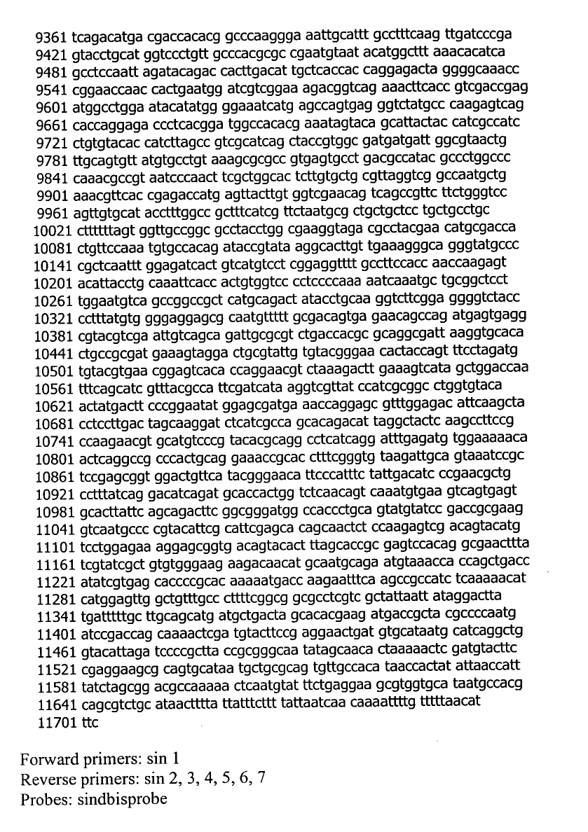

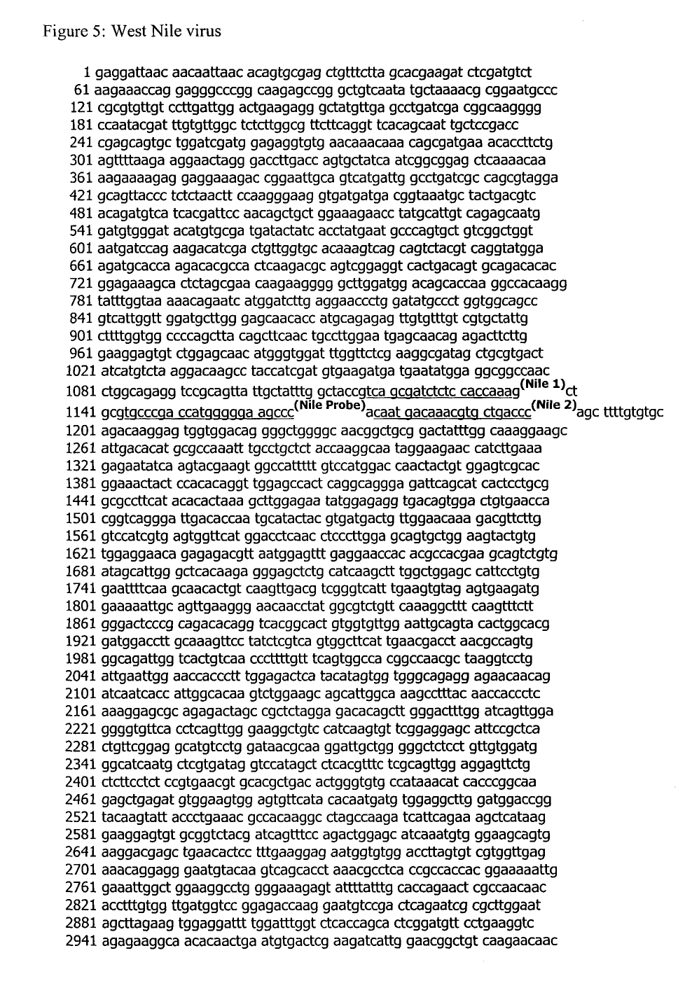

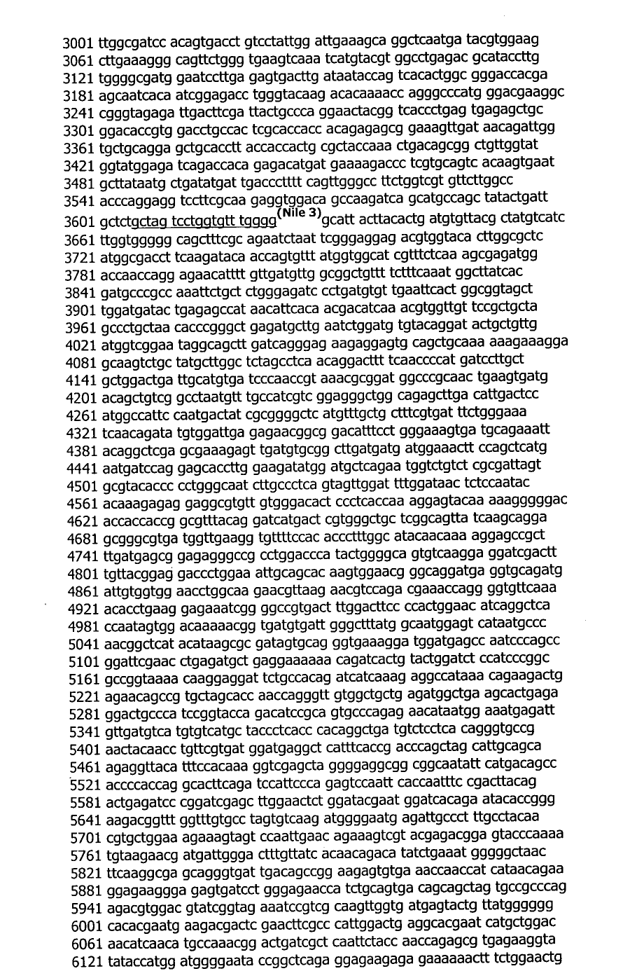

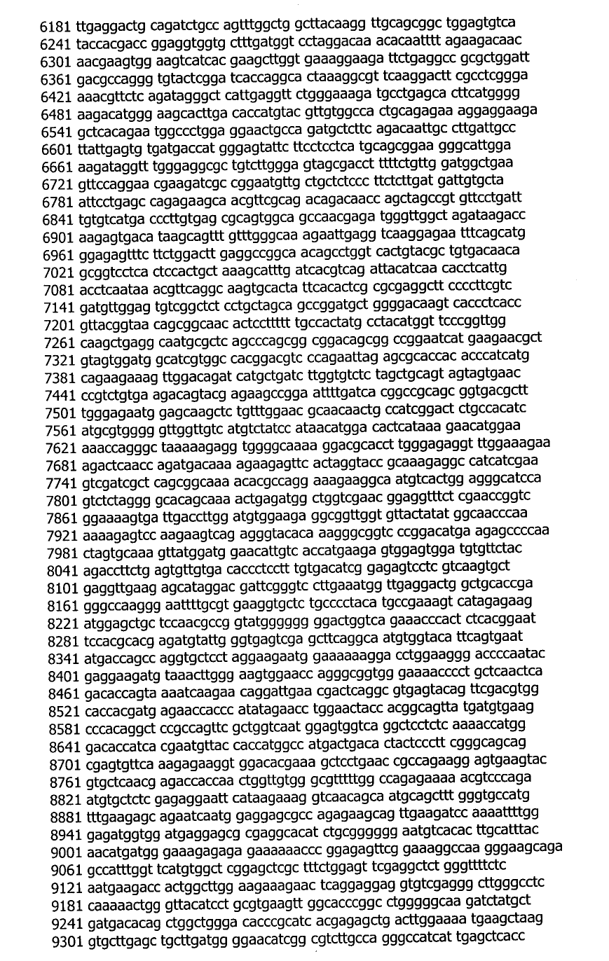

5. Primers: Prism 11 and Prism 12 (FIG. 3); and

6. Probe 6 (FIG. 3).

Procedure: 1. To 100 μl viral sample, added 50 μl tris-HCl buffer, 60 μl protein degrader, and 200 μl cell lysis buffer;

2. Mixed and incubated for 25 minutes (5 minutes at 70° C.);

3. Diluted samples to 1/50, 1/500, 1/5000, 1/25000, 1/50000, 1/250000 and 1/500000;

4. Ran PCR for 55 cycles.

Results: Results showed that unirradiated material had regular dilution series curves, irradiated material (50 kGy) behaved differently, dilute material did not amplify showing a reduction in the number of copies of the target sequence.

### Example 3

Purpose: To determine effects of gamma irradiation (0 kGy sample, 50 kGy sample, mixture of 0+50 kGy sample and 75 kGy sample) on samples containing PPV analyzed by PCR.

Materials: 1. PPV (irradiated at 0 kGy, 50 kGy or 75 kGy);

2. Primers: Prism 11 & Prism 12, Probe 6 (FIG. 3);

3. Primers: Prism 1 & Prism 2, Probe 1 (FIG. 3).

Procedure: 1. Diluted samples containing PPV to 1/100, 1/1000, 1-2000, 1/10000, 1/20000, 1/40000 and 1/400000 (0 kGy, 50 kGy, 0+50 kGy and 75 kGy); 2. Ran PCR program for 55 cycles.

Results: Irradiaition to 50 kGy of PPV material reduced amplification of 549 bp amplicon.

### Example 4

Purpose: To examine the relative effectiveness of Qiagen and Taqman reagents on samples containing PPV.

Materials: 1. PPV DNA (phenol extracted);

2. Taq PCR Core Kit;

3. ProofStart DNA polymerase;

4. Taqman Universal PCT Master Mix;

5. Prism 1, 2, 11 and 17 (FIG. 3);

6. Probes 1 and 6 (FIG. 3);

7. Agarose;

8. TAE;

9. EtBr.

Procedure: 1. Prepared the following four master mixes:

| a. Qiagen: | 1 | 2 |
|---|---|---|
| 10x buffer: | 30 μl | 25 μl |
| dNTP's: | 9 μl | 7.5 μl |
| pA: | 8.34 μl | 6.95 μl |
| pB: | 8.34 μl | 6.95 μl |
| taq: | 6 μl | 5 μl |
| H₂O: | 187.32 μl | 156.1 μl |
| probe: | 15 μl | 12.5 μl |
| b. Taqman: | 3 | 4 |
| Master Mix: | 150 μl | 125 μl |
| pA: | 15 μl | 12.5 μl |
| pB: | 15 μl | 12.5 μl |
| probe: | 15 μl | 12.5 μl |
| H₂O: | 69 μl | 57.5 μl |

2. Pipetted 44 μl of master mix 1 into row D, wells 1 and 2; row E, wells 1 and 2; and row H, well 1, of a well plate;

3. Pipetted 44 μl of master mix 2 into row D, wells 3 and 4; and row E, wells 3 and 4, of a well plate;

4. Pipetted 44 μl of master mix 3, into row F, wells 1 and 2; row G, wells 1 and 2; and row H, well 3, of a well plate;

5. Pipetted 44 μl of master mix 4 into row F, wells 3 and 4; and row G, wells 3 and 4, of a well plate;

6. Added 1 μl of ProofStart taq to row D, wells 1-4 and row F, wells 1-4 and added 1 μl water to remaining wells;

7. Added 5 μl water to row H, wells 1 and 3 and added 5 μl PPV DNA to remaining wells;

8. Ran PCR for 40 cycles.

Results: Qiaqen Master with ProofStart taq produced functional large amplicons in realtime PCR with PPV DNA more efficiently than the TaqMan master mix.

### Example 5

Purpose: To examine the effects of proofstart in amplifying large amplicons and to examine the effects of 50 kGy irradiation on PPV.

Materials: 1. PPV DNA (irradiated to 0 kGy and 50 kGy);

2. Taq PCR Core Kit;

3. Proofstart DNA polymerase;

4. Prism 11, 16 and 17 (FIG. 3);

5. Agarose;

6. Ethidium Bromide;

7. TAE buffer.

Procedure: 1. Set up PCR master mix as follows:

| | | |
|---|---|---|
| 10x buffer: | 50 | μl |
| dNTP's: | 15 | μl |
| pA: | 13.9 | μl (primer 11) |
| taq: | 10 | μl |
| water: | 347.2 | μl |

2. Placed aliquots into PCR tubes;

3. Added either primer 16 or 17 to PCR tubes;

4. Added PPV DNA (diluted to 1:100) to each PCR tube:

5. Added 10 μl proofstart to half of the samples (2 at 0 kGy and 2 at 50 kGy);

6. Performed PCR (about 55 cycles)

7. Poured a 1% gel and ran at 100 V for 20 minutes.

Results: Addition of a proofreading polymerase resulted in improved amplication of longer amplicons. Delay in amplification of target sequence in irradiated samples is proportional to damage done to viral genetic material.

### Example 6

Purpose: To examine the effect of TSP concentration on amplification of large target amplicons in gamma irradiated and unirtadiated PPV.

Materials: 1. TSP (cat. no. M02965);

2. Qiagen Core kit;

3. ProofStart DNA polymerase;

4. PPV (irradiated to 0 kGy or 50 kGy).

Procedure: 1. Prepared a master mix (standard PCR set-up) for each (TSP: Taq 1:20, 1:10, 1:5);

2. Added 43.61 μl of each master mix (TSP titration) to PCR tubes;

3. Added 1.39 μl of primers 16, 17 or 19 (FIG. 3) to appropriate PCT tubes;

4. Added 5 μl water to the negative control, which contained primer pair 11, 16 (FIG. 3).

5. Diluted PPV 1:100;

6. Added PPV to PCR tubes;

7. Performed PCR;

8. Poured a 1% gel and ran at 100 V for 20 minutes.

Results: Under these conditions, addition of TSP resulted in increased amplification of target amplicons in both irradiated and unirradiated samples, but irradiation of PPV resulted in decreased amplification of target amplicon.

### Example 7

Purpose: To examine the effects of gamma irradiation on amplification of PPV target amplicons of various sizes.

Materials: 1. PPV DNA (irradiated to 0 kGy or 50 kGy);

2. Taq PCR Core Kit;

3. ProofStart DNA Polymerase;

4. Prism 11, 16, 17, 18 and 19 (FIG. 3);

5. Agarose;

6. TAE;

7. Ethidium Bromide.

Procedure: 1. Prepared PCR Master Mix as follows:

| | |
|---|---|
| 10x Buffer | 5 μl |
| dNTPs | 1.5 μl |
| pA | 1.39 μl |
| pB | 1.39 μl |
| taq | 1 μl |
| water | 33.72 μl |
| PPV | 5 μl. |

2. Alliquoted samples into PCR tubes;

3. Ran PCR;

4. Poured a 1% agarose gel and ran at 120 V for about 1.5 hours.

Results: Irradiation to 50 kGy resulted in decreased amplification of larger target amplicons.

### Example 8

Purpose: To examine PCR sensitivity and determine log reduction of PPV in samples irradiated to 50 kGy and having a starting concentration of 2.5×10⁷ gEq.

Materials: 1. Standard PCR reagents (Qiacen Core Kit, TSP, Proofstart, etc.);

2. Primers 11 and 17 (FIG. 3);

3. PPV extract.

Procedure: 1. Prepared master mix with primers 11 and 17;

2. Performed a 10 fold dilution series from 10⁷ to 10⁰ of PPV extract;

3. Pipetted 45 μl of master mix into PCR tubes;

4. Pipetted 5 μl of each PPV dilution into appropriated PCR tubes;

5. Added 5 μl water to control;

6. Ran PCR;

7. Ran samples in 1% agarose at 100V for about 47 minutes.

Results: Irradiation of sample to 50 kGy resulted in decreased amplification of target amplicon across all concentration ranges.

### Example 9

Purpose: To examine PCR sensitivity and determine log reduction of PPV in samples irradiated to 50 kGy and having a starting concentration of 2.5×10⁷ gEq.

Materials: 1. TSP;

2. Standard PCR kit (Qiacen with ProofStart Polymerase);

3. Primers 11 and 19 (FIG. 3);

4. PPV Extract (Irradiated to 0 kGy and 50 kGy).

Procedure: 1. Prepared master mix with primers 11 and 19;

2. Performed a 10 fold dilution series from 10⁷ to 10⁰ of PPV extract;

3. Pipetted 45 μl of master mix into PCR tubes;

4. Pipetted 5 μl of each PPV dilution into appropriated PCR tubes;

5. Added 5 μl water to control;

6. Ran PCR as follows: 95° C. for 2 minutes (1 cycles)

94° C. for 10 seconds (40 cycles)

60° C. for 1 minute (40 cycles)

68° C. for 2 minutes (40 cycles);

7. Cooled to 4° C.;

8. Ran samples on 1% agarose gel in 1×TAE and 5 μl/100 ml ethidium bromide at 100 V for 52 minutes (5 μl on gel).

Results: Irradiation to 50 kGy resulted in decreased amplification of target amplicon across all concentration ranges. For unirradiated samples, relative band strength of observed target amplicon decreased with decreasing concentration.

### Example 10

Purpose: Primer validation for B19 using probe 7 and various primers.

Materials: 1. B19 IGIV Paste (irradiated to 0 kGY or 50 kGy);

2. EXB;

3. Proteinase;

4. yeast tRNA

5. phenol chloroform isoamyl alcohol;

6. 3M NaAc;

7. isopropanol;

8.70% EtOH;

9. TE buffer;

10. Prisms 5, 6, 20, 21, 22, 23, 24, 25, 26 (FIG. 1);

11. Qiagen reagents;

12. Ampligold Taq;

13. ProofStart Polymerase;

14. Agarose;

15. TAE;

16. Ethidium Bromide.

Procedure: 1. Prepared a Master Mix as follows:

| | |
|---|---|
| Buffer | 5 μl |
| DNTP | 1.5 μl |
| Taq | 1 μl |
| DNA | 5 μl |
| water | 34.72 μl |

2. Pipetted Master Mix into PCR tubes;

3. Added the following primer pairs to appropriate PCR tubes: 20&21; 20&22; 20&23; 20&24; 20&25; 20&6; 20&26; 5&6;

4. Ran PCR;

5. ran 1% gel for about 1 hour.

Results: All tested primers yielded desired target amplicons.

### Example 11

Purpose: Use of PCR multiplexing with target amplicons of about 112 bp and about 2.4 kbp for B19 virus in samples irradiated to 0 kGy or 50 kGy.

Materials: 1. TSP thermostable inorganic pyrophosphatase

2. Standard PCR reagents;

3. B19 viral extract (irradiated to 0 kGy and 50 kGy);

4. Prisms 5, 6, 20 and 25 (FIG. 1);

5. Taq;

6. ProofStart Polymerase.

Procedure: 1. Prepared standard PCR set-up with 3× master mixes, for each primer set (primer sets: 5&6; 20&25; 5&6; and 20&25);

2. Prepared appropriate PCR tubes containing the following primer pairs: (5, 6) 0 kGy; (5, 6) 50 kGy; (20, 25) 0 kGy; (20, 25) 50 kGy; (5, 6) & (20, 25), 0 kGy; and (5, 6) and (20, 25), 50 kGy;

3. Added 5 μl B19 to PCR tubes containing 45 μl of appropriate master mix;

4. Added 5 μl water to control;

5. Ran PCR.

6. Ran samples on 1% aragose gel at 100 V for about 17 minutes.

Results: PCR multiplexing is effective for mixtures containing large target amplicons and small target amplicons. Irradiation to 50 kGy resulted in decreased amplification of the large target amplicon relative to the small target amplicon.

### Example 12

Purpose: Irradiated and unirtadiated samples containing B19 viral material were examined using real time PCR.

Materials: 1. B19 viral material (irradiated to 0 kGy and 50 kGy);

2. Prism pairs (20, 21) and (20, 26) (FIG. 1);

3. Qiagen PCR reagents;

4. Qiagen ProofStart;

5. Agarose;

6. TAE (1×);

7. sample loading buffer (SLB).

Procedure: 1. Prepared standard samples containing primer pairs with 10¹¹ to 10¹ dilution series;

2. Ran PCR (40 cycles);

3. Ran gel on 1% agarose (8 μl PCR product, 1 μl SLB) at 100 V for about 20 minutes.

Results: Unirradiated and irradiated samples amplified in a regular pattern for a dilution series with a small amplicon. As amplicon size increased, unirradiated material maintained a regular dilution pattern while irradiated material did not.

### Example 13

Purpose: To investigate the effect of gamma irradiation on samples containing HBV clone and irradiated to 50 kGy.

Materials: 1. HBV (irradiated to 0 kGy and 50 kGy);

2. Taq PCR Core Kit;

3. ProofStart DNA polymerase;

4. Prisms 3, 4, 9, 10, 15, 29, 30, 31, 36 and 37;

5. Agarose;

6. TAE Buffer;

7. ethidium bromide.

Procedure: 1. Prepared PCR master mix as follows:

| | |
|---|---|
| 10x PCR buffer | 5 μl |
| dNTPs | 1.39 μl |
| primers | 1.39 μl |
| taq | 1 μl |
| ProofStart | 1 μl |
| water | 33.22 μl |
| TSP | 0.5 μl |

2. Aliquoted 43.61 μl of master mix into PCR tubes. Appropriate tubes contained the following primer pairs: (3, 4); (9, 10); (9, 15); (9, 29); (9, 30); (9, 31); (36, 37); and (9, 31), for both irradiated and unirradiated samples;

3. Added 5 μl HBV per tube (irradiated or unirradiated);

4. Ran PCR as follows:

50° C. for 2 minutes (one cycle)

95° C. for 2 minutes (one cycle)

94° C. for 10 seconds (40 cycles)

60° C. for 1 minute (40 cycles)

68° C. for 2 minutes, five seconds (40 cycles);

5. Ran 1% agarose gel (9 μl sample+1 μl sample buffer) at 100 v for about 20 minutes.

Results: Irradiated samples showed no band, indicating degradation of HBV clone by irradiation to 50 kGy.

### Example 14

Purpose: To investigate the effect of gamma irradiation on samples containing HBV DNA and irradiated to 50 kGy.

Materials: 1. HBV DNA material (irradiated to 0 kGy and 50 kGy);

2. Taq PCR Core Kit (Qiagen, cat. no. 201223);

3. ProofStart Taq Polymerase (Qiagen, cat. no. 20);

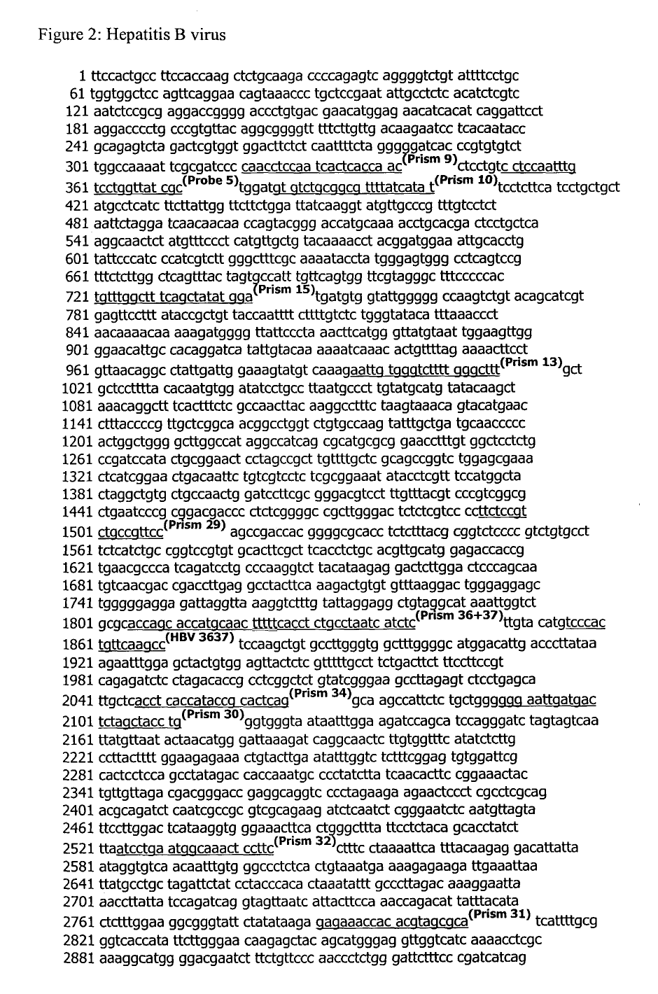

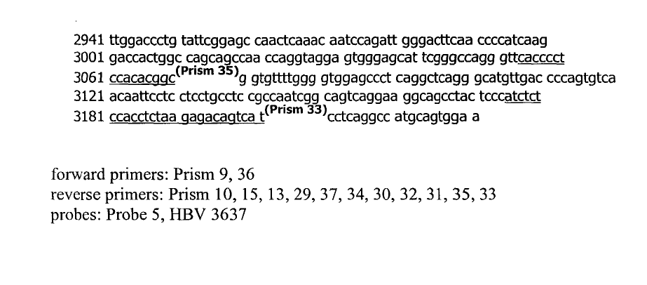

4. Prisms 10, 13, 30, 36 and 37 (FIG. 2);

5. Agarose;

6. TAE Buffer;

7. Ethidium Bromide.

Procedure: 1. Prepared the following master mix:

| | |
|---|---|
| 10x buffer | 60 μl |
| dNTP | 18 μl |
| primer 36 | 16.68 μl |
| Taq | 12 μl |
| ProofStart | 12 μl |
| water | 440.64 μl; |

2. Pipetted 46.61 μl of master mix into PCR tubes;

3. Added 1.39 μl of reverse primer (10, 13, 30 or 37) and 2 μl HBV DNA (0 kGy and 50 kGy) to appropriate tubes;

4. Ran PCR for 50 cycles;

5. Poured a 1% agarose gel (8 μl PCR product+1 μl sample buffer) at 100 V for about 20 minutes.

Results: Irradiated samples showed no band, indicating degradation of HBV DNA by irradiation to 50 kGy.

### Example 15

Purpose: HBV amplification of nested primer set (about 80 bp, 400 bp and 697 bp) in samples containing ascorbate, including digestion of 0 kGy and 50 kGy samples with exonuclease I prior to PCR amplication.

Materials: 1. HBV DNA (irradiated to 0 kGy and 50 kGy, with and without ascorbate);

2. Primer sets: (9, 10); (9, 14) and (9, 13) (FIG. 2);

3. Exonuclease I;

4. Standard PCR reagents.

Procedure: 1. Diluted HBV samples to 1/500, 1/2000 and 1/10000;

2. Digested 1 μl raw HBV extract in 0.25 μl Exonuclease I, 10 μl 10× Exonuclease I buffer and 88.75 μl water at 37° C. for 30 minutes, inactivated at 80° C. for 20 minutes;

3. Dilutes digested HBV to 1/2000 and 1/10000;

4. Ran 55 cycles PCR.

Results: Irradiated and unirradiated samples coamplified with an 80 bp product. Only unirradiated samples amplified with a 697 bp product.

### Example 16

Purpose: To investigate the amount of bacterial and fungal DNA present in pulverized tendon samples.

Materials: 1. E. Coli samples (tendon)−0 or 50 kGy+stabilizer (6.65×1¹⁰ CFU/μl);

2. C. Albicans samples (tendon)−0 or 50 kGy+stabilizer (3.55×10⁹ CFU/μl);

3. Staph. Aureus samples;

4. Control tendon;

5. Dneasey tissue kit (Qiagen, cat. no. 69504);

6. Taq PCR Core Kit (Qiagen, cat. no. 201223);

7. ProofStart Taq Polymerase (Qiagen, cat. no. 202205);

8. Primers: Ribo 7, 8, 10, 11, 12, 13, 14 and Fungi 1, 2, 3, 4, 5, 6, 7, 8 (FIGS. 6A and 6B);

9. Probes: FAM-RIBO Fungi Probe 5′-6FAM-TACAGTGAAACTGCGAATGGCTCATTAAATCAGTTA-TAMRA-3′;

10. Microcon YM Centrifugal Filter Unit;

Procedure: 1. Using 0.05 tendon samples for E. coli and C. albicans, followed the Qiagen extraction profile;

2. Prepared the following master mixes:

| | Mix 1 | Mix 2 |
|---|---|---|
| 10x buffer | 150 μl | 85 μl |
| dNTPs | 45 μl | 25.5 μl |
| Ribo 741.7 | | — |
| Fungi 1 | — | 23.65 μl |
| Taq | 30 μl | 17 μl |
| ProofStart | 30 μl | 17 μl |
| Water | 936.6 μl | 530.74 μl |
| FAM-RIBO | 75 μl | — |
| Fungi Probe | — | 42.5 μl |

3. Filtered master mixes using Microcon filter units for 30 minutes at 100×g;

4. Pipetted 43.6 μl of Mix 1 into: rows A-D, columns 1-6; rows A-C, column 9; and row E, column 12;

5. Pipetted 43.6 μl of Mix 2 into: rows E-F, columns 1-7 and row H, column 12;

6. Pipetted 1.39 μl of reverse primer into appropriate well;

7. Pipetted 5 μl DNA into appropriate wells;

8. Ran PCR.

Results: Irradiation with 50 kGy resulted in decreased amplification of large target amplicons, indicating degradation of the pathogen genetic material caused by irradiation.

### Example 17

Purpose: To show functionality of E. coli primers for RT-PCR using large target amphlicons.

Materials: 1. E. coli prepared from overnight culture;

2. Dneasy Tissue Kit (Qiagen, cat. no. 96504); 3. Taq PCR Core Kit (Qiagen, cat. no. 201223)

4. PtoofStart DNA polymerase (Qiagen, cat. no. 202205);

5. Microcon YM-100 Centrifugal Filter Unit (cat. no. 42413);

6. Primers: Ribo 1-9;

7. Agarose;

8. TAE Buffer;

9. Ethidium Bromide.

Procedure: 1. Pipetted 1 ml of E. coli culture into each of 10 1.5 tubes;

2. Centrifuged all 10 tubes for 5 minutes at maximum speed;

3. Discarded supernatant;

4. Placed 8 tubes in −80° C. and used 2 tubes for extraction following the Qiagen protocol;

5. Prepared Master Mix as follows:

| | | |
|---|---|---|
| 10x Buffer | 5 | μl |
| dNTPs | 1.5 | μl |
| pA | 1.39 | μl (Ribo 1 or 7) |
| pB | 1.39 | μl (Ribo 2, 3, 4, 5, 6, 8 or 9) |
| Taq | 1 | μl |
| ProofStart | 1 | μl |
| Water | 33.22 | μl |
| TSP | 0.5 | μl |

6. Mixed Master Mix by inversion;

7. Pipetted Master mix into a Microcon Centrifugal Filter Unit and centrifuged for 30 minutes at 100×g;

8. Pipetted 43.61 μl of Master Mix into PCR tubes;

9. Added appropriate reverse primer and DNA or water to create the following primer pairs: (1, 2)+5 μl DNA; (1, 2)+1 μl; (1, 3)+5 μl DNA; (1, 3)+1 μl DNA; (1, 4)+5 μl DNA; (1, 4)+1 μl DNA; (1, 5)+5 μl DNA; (1, 5)+1 μl DNA; (1, 6)+5 μL DNA; (1, 6)+1 μl DNA; (5, 8)+5 μl DNA; (7, 8)+1 μl DNA; (7, 9)+5 μl DNA; (7, 9)+1 μl DNA; and (1, 2)+5 (1, 4)+5 μl water;

10. Ran PCR;

11. Ran 1% Agarose gel at 100 V for about 20 min.

Results: All E. coli primers showed amplification of target sequences, regardless of size.

### Example 18

Purpose: To investigate the effects of 50 kGy irradiation on samples containing E. coli.

Materials: 1. E. coli spiked tendon (irradiated to 0 kGy and 50 kGy)+6.65×10¹⁰ CFU/μl;

2. Taq PCR Core Kit (Qiagen, cat. no. 201223);

3. ProofStart Taq Polymerase (Qiagen, cat. no. 202205);

4. Primers: Ribo 7, 8, 13, 14 and 15;

5. Agarose;

6. TAE Buffer;

7. Ethidium Bromide;

8. Microcon Centrifugal Filter Unit.

Procedure: 1. Prepared Master Mix as follows:

| | |
|---|---|
| 10x Buffer | 60 μl |
| dNTP | 18 μl |
| pA (forward) | 16.68 μl |
| Taq | 12 μl |
| ProofStart | 12 μl |
| Water | 452.64 μl; |

2. Placed in Microcon and centrifuged for 30 minutes at 100×g;

3. Pipetted 47-61 μl master mix into each or 9 PCR tubes;

4. Added 1.39 μl of reverse primer and 1 μl DNA into appropriate tubes;

5. Ran PCR.

6. Ran 1% Agarose gel (8 μl sample+1 μl sample buffer) at 100 V for about 20 minutes.

Results: Samples irradiated to 50 kGy showed progessive disappearance of bands with increasing amplicon size, indicating degradation of the E. coli genetic material caused by irradiation.

### Example 19

Purpose: To show functionality of Mt-DNA primers for RT-PCR using large target amplicons.

Materials: 1. Tendon DNA (irradiated to 0 kGy and 50 kGy);

2. ROX 6 (1/10 dilution) molecular probes;

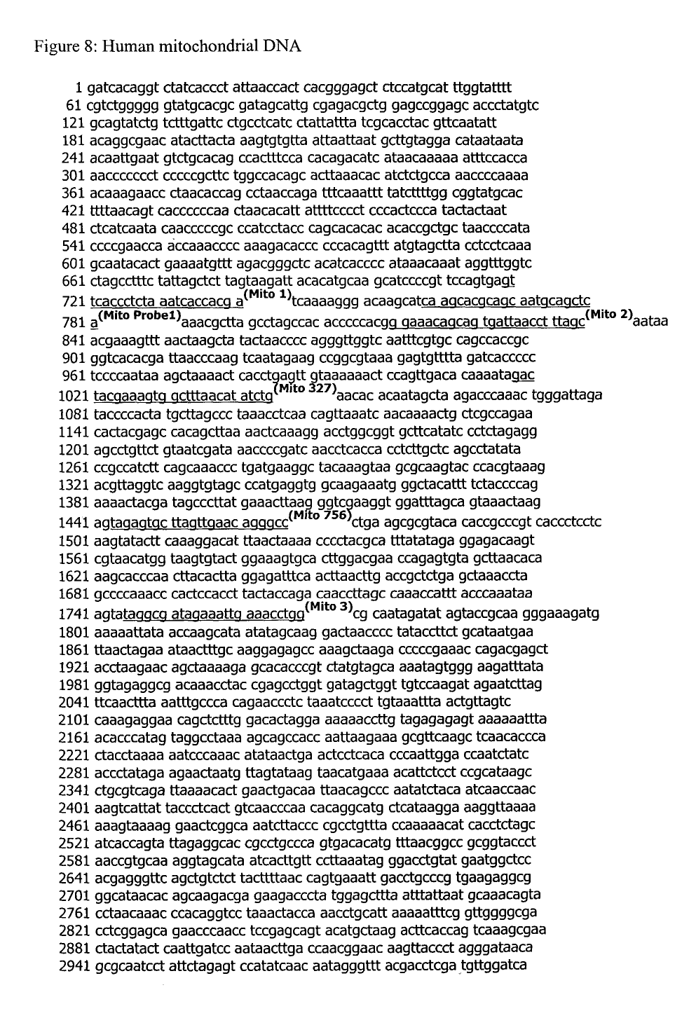

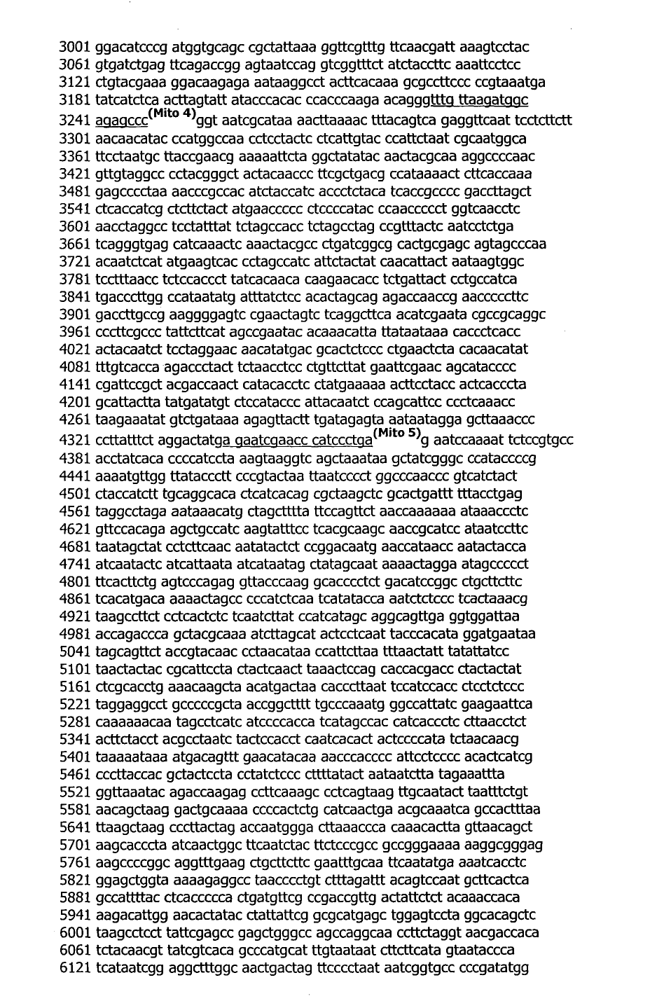

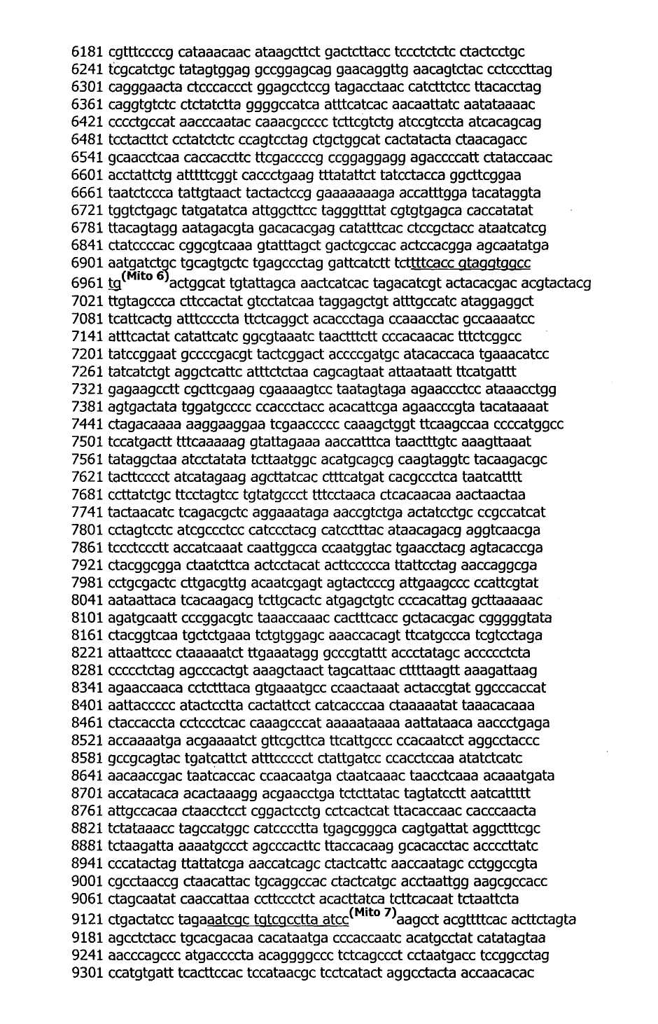

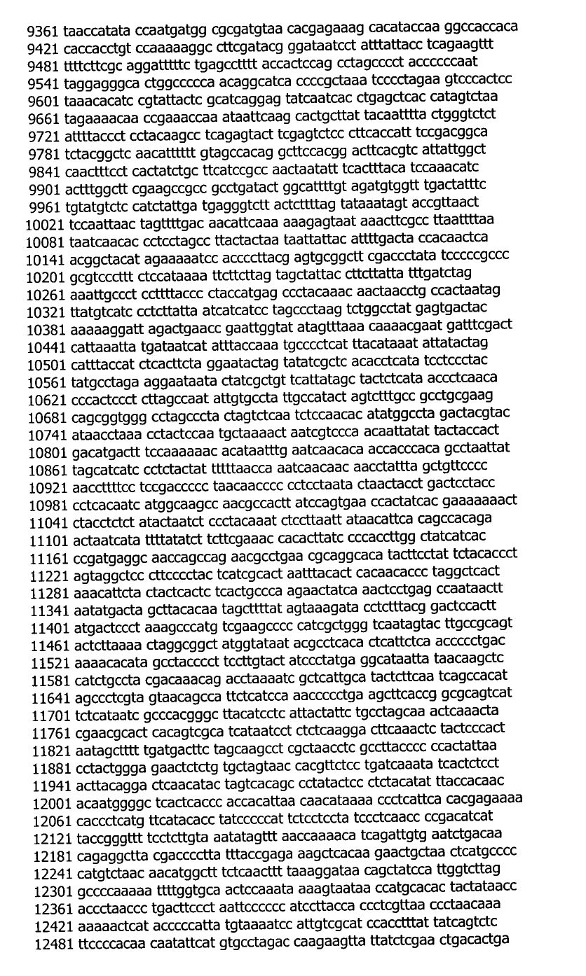

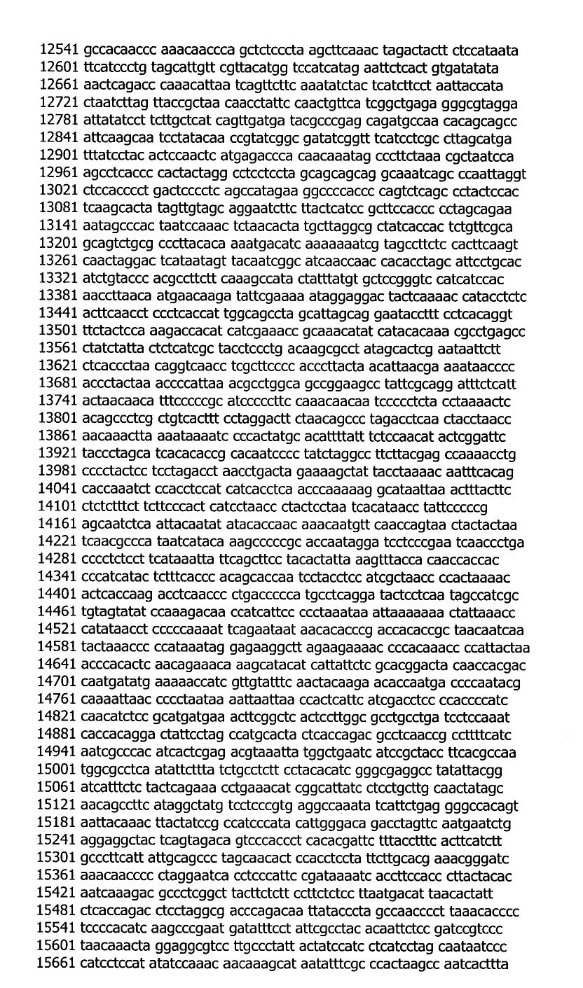

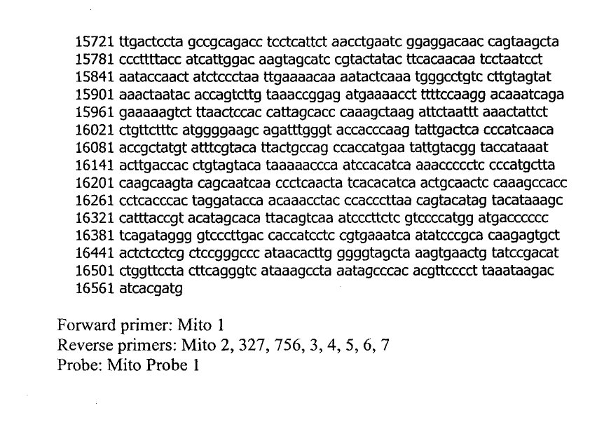

3. Primers: MITO 1, 2, 3, 4, and 5 (FIG. 8);

4. MITO Probe 1 (FIG. 8);

5. Human DNA;

6. Qiagen PCR Reagants;

7. Qiagen ProofStart.

Procedure: 1. Prepared the following mixtures:

| | |
|---|---|
| Buffer | 1.5 μl |
| dNTPs | 1.5 μl |
| MITO 1 | 2.5 μl |
| reverse primer | 2.5 μl (MITO 2, 3, 4 or 5) |
| MITO Probe | 2.5 μl |
| Taq | 1 μl |
| PS | 1 μl |
| 1/10 ROX | 1 μl |
| water | 28 μl |
| DNA | 5 μl |

2. Ran 40 PCR;

3. Ran 1% agatose gel (8 μl product+1 μl sample loading buffer) at 100 V for about one hour.

Results: Mt-DNA primers were functional, regardless of amplicon size.

Having now fully described this invention, it will be understood to those of ordinary skill in the art that the methods of the present invention can be carried out with a wide and equivalent range of conditions, formulations and other parameters without departing from the scope of the invention or any embodiments thereof. Moreover, the methods of the present invention may also be applied to situations other than the preferred embodiments described above. For example, instead of determining the level of potentially active pathogens, the methods described above may be used to determine the number of cells having an altered genetic sequence, such as turnout cells or genetically modified cells, in a tissue sample.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

What is claimed is:

1. A method for determining the level of potentially active biological pathogens in a biological material, said method comprising:
(i) adding to said biological material an effective amount of at least two nucleic acid primer pairs,
wherein a first nucleic acid primer pair hybridizes under stringent conditions to a first target nucleic acid sequence found in said biological pathogen and a second nucleic acid primer pair hybridizes under stringent conditions to a second target nucleic acid sequence found in said biological pathogen, and
further wherein said first target nucleic acid sequence and said second target nucleic acid sequence are not identical and said second target nucleic acid sequence contains more nucleic acid residues than said first target nucleic acid sequence;
(ii) amplifying said target nucleic acid sequences by polymerase chain reaction, said polymerase chain reaction comprising adding at least one polymerase to said biological material containing said nucleic acid primer pairs to form an amplification mixture and thermally cycling said amplification mixture between at least one denaturation temperature and at least one elongation temperature for a period of time sufficient to amplify said target nucleic acid sequences; and
(iii) detecting and quantifying said first and second target nucleic acid sequences, wherein the quantity of said first target nucleic acid sequence is proportional to the number of said biological pathogens in said biological material and the quantity of said second target nucleic acid sequence is proportional to the number of potentially active biological pathogens in said biological material.

2. The method according to claim 1, wherein said first target nucleic acid sequence contains between about 50 and about 500 nucleic acid residues

3. The method according to claim 1, wherein said second target nucleic acid sequence contains between about 500 and about 50,000 nucleic acid residues.

4. The method according to claim 1, wherein the nucleic acid sequence of said first target nucleic acid sequence and the nucleic acid sequence of said second target nucleic acid sequence contain at least 16 contiguous nucleic acid residues in common.

5. The method according to claim 1, wherein said step (i) further comprises adding at least one nucleic acid probe to said biological material.

6. The method according to claim 5, wherein said nucleic acid probe is selected from the group consisting of 5′ nuclease probes, hairpin probes, adjacent probes, sunrise probes and scorpion probes.

7. The method according to claim 1, wherein, prior to step (i), said biological material has been subjected to a process that alters at least one wild-type nucleic acid sequence in said biological pathogen.

8. The method according to claim 7, wherein said process comprises irradiating said biological material with gamma radiation.

9. The method according to claim 1, wherein one of said first pair of nucleic acid primers and one of said second pair of nucleic acid primers are substantially identical.

10. The method according to claim 1, wherein neither of said first pair of nucleic acid primers is substantially identical to either of said second pair of nucleic acid primers.

11. The method according to claim 1, wherein said first pair of nucleic acid primers and said second pair of nucleic acid primers are substantially identical.

12. The method according to claim 5, wherein said nucleic acid probe contains at least 16 nucleic acid residues.

13. The method according to claim 1, wherein said biological pathogen is selected from the group consisting of bacteria, viruses, fungi and single cell parasites.

14. The method according to claim 1, wherein said first target nucleic acid sequence is at least 30% homologous to a wild-type nucleic acid sequence found in said biological pathogen.

15. The method according to claim 1, wherein said second target nucleic acid sequence is at least 30% homologous to a wild-type nucleic acid sequence found in said biological pathogen.

16. The method according to claim 14 or 15, wherein said biological pathogen is selected from the group consisting of Aspergillus, Candida, Histoplasma, Saccharomyces, Coccidioides and Cryptococcus.

17. The method according to claim 14 or 15, wherein said biological pathogen is selected from the group consisting of Escherichia, Bacillus, Campylobacter, Helicobacter, Lysteria, Clostridium, Streptococcus, Enterococcus, Staphylococcus, Brucella, Haemophilus, Salmonella, Yersinia, Pseudomonas, Serratia, Enterobacter, Kebsiella, Proteus, Citrobacter, Corynebacterium, Propionibacterium and Coxiella.

18. The method according to claim 14 or 15, wherein said biological pathogen is selected from the group consisting of Adeno-associated Virus (AAV), California Encephalitis Virus, Coronavirus, Coxsackievirus-A, Coxsackievirus-B, Eastern Equine Encephalitis Virus (EEEV), Echovirus, Hantavitus, Hepatitis A Virus (HAV), Hepatitis C Virus (HCV), Hepatitis Delta Virus (HDV), Hepatitis E Virus (HEV), Hepatitis G Virus (HGV), Human Immunodeficiency Virus (HIV), Human T-lymphotrophic Virus (HTLV), Influenza Virus (Flu Virus), Measles Virus (Rubeola), Mumps Virus, Norwalk Virus, Parainfluenza Virus, Polio virus, Rabies Virus, Respiratory Syncytial Virus, Rhinovirus, Rubella Virus, Saint Louis Encephalitis Virus, Western Equine Encephalitis Virus (WEEV), Yellow Fever Virus, Adenovirus, Cytomegalovirus (CMV), Epstein-Barr Virus (EBV), Hepatitis B Virus (HBV), Herpes Simplex Virus 1 (HHV1), Herpes Simplex Virus 2 (HHV2), Molluscum contagiosum, Papilloma Virus (HPV), Smallpox Virus (Variola), Vaccinia Virus, Venezuelan Equine Encephalitis Virus (VEEV), Ebola Virus, West Nile Virus, Human Parvovirus B19 and Rotavirus.

19. The method according to claim 15, wherein said wild-type nucleic acid sequence comprises the 16S ribosomal RNA gene coding sequence.

20. The method according to claim 15, wherein said wild-type nucleic acid sequence comprises the 16S ribosomal RNA gene coding sequence and a portion of the 23S ribosomal RNA gene coding sequence.

21. The method according to claim 15, wherein said wild-type nucleic acid sequence comprises the 16S ribosomal RNA gene coding sequence and a portion of the 23S ribosomal RNA gene coding sequence and the non-coding sequence therebetween.

22. The method according to claim 14, wherein said wild-type nucleic acid sequence comprises the 18S ribosomal RNA gene coding sequence.

23. The method according to claim 14, wherein said wild-type nucleic acid sequence comprises the 18S ribosomal RNA gene coding sequence and the 5.8S ribosomal RNA gene coding sequence.

24. The method according to claim 14, wherein said wild-type nucleic acid sequence comprises the 18S ribosomal RNA gene coding sequence and the 5.8S ribosomal RNA gene coding sequence and the non-coding sequence therebetween.

25. The method according to claim 14, wherein said wild-type nucleic acid sequence comprises the 18S ribosomal RNA gene coding sequence and the 5.8S ribosomal RNA gene coding sequence and a portion of the 28S ribosomal RNA gene coding sequence.

26. The method according to claim 14, wherein said wild-type nucleic acid sequence comprises the 18S ribosomal RNA gene coding sequence and the 5.8S ribosomal RNA gene coding sequence and a portion of the 28S ribosomal RNA gene coding sequence and the non-coding sequences therebetween.

27. The method according to claim 1, wherein said polymerase is a thermostable polymerase.

28. The method according to claim 27, wherein said thermostable polymerase is a Taq polymetase.

29. The method according to claim 1, wherein said polymerase chain reaction thermally cycling said amplification mixture between at least one denaturation temperature, at least one annealing temperature and at least one elongation temperature for a period of time sufficient to amplify said target nucleic acid sequence.

30. The method according to claim 7, wherein said process fragments said at least one wild-type nucleic acid sequence in said biological pathogen.

31. The method according to claim 7, wherein said process cross-links said at least one wild-type nucleic acid sequence in said biological pathogen.

32. The method according to claim 7, wherein said process covalently modifies said at least one wild-type nucleic acid sequence in said biological pathogen.

33. The method according to claim 1, wherein said biological material is selected from the group consisting of: cells; tissues; blood or blood components; proteins; enzymes; immunoglobulins; botanicals; and food.

34. The method according to claim 1, wherein said biological material is selected from the group consisting of ligaments; tendons; nerves; bone; teeth; skin grafts; bone marrow; heart valves; cartilage; corneas; arteries and veins; organs; lipids; carbohydrates; collagen; chitin and its derivatives; forensic samples, mummified material; human or animal remains; stem cells; islet of Langerhans cells; cells for transplantation; red blood cells; white blood cells; and platelets.


SEQUENCE LISTING (99 sequences)

SEQ ID NO: 1 - DNA, 5594 bp, B19 virus
ccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcttgcttggggttgccttgacactaagacaagcggcgcgccgcttgtcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaattttagcgggcttttttcccgccttatgcaaatgggcagccattttaagtgtttcactataattttattggtcagttttgtaacggttaaaatgggcggagcgtaggcggggactacagtatatatagcacggcactgccgcagctctttctttctgggctgctttttcctggactttcttgctgttttttgtgagctaactaacaggtatttatactacttgttaacatactaacatggagctatttagaggggtgcttcaagtttcttctaatgttctggactgtgctaacgataactggtggtgctctttactggatttagacacttctgactgggaaccactaactcatactaacagactaatggcaatatacttaagcagtgtggcttctaagcttgactttaccggggggccactagcggggtgcttgtacttttttcaagtagaatgtaacaaatttgaagaaggctatcatattcatgtggttattggggggccagggttaaaccccagaaacctcacagtgtgtgtagaggggttatttaataatgtactttatcaccttgtaactgaaaatgtaaagctaaaatttttgccaggaatgactacaaaaggcaaatactttagagatggagagcagtttatagaaaactatttaatgaaaaaaatacctttaaatgttgtatggtgtgttactaatattgatggatatatagatacctgtatttctgctacttttagaaggggagcttgccatgccaagaaaccccgcattaccacagccataaatgacactagtagtgatgctggggagtctagcggcacaggggcagaggttgtgccaattaatgggaagggaactaaggctagcataaagtttcaaactatggtaaactggttgtgtgaaaacagagtgtttacagaggataagtggaaactagttgactttaaccagtacactttactaagcagtagtcacagtggaagttttcaaattcaaagtgcactaaaactagcaatttataaagcaactaatttagtgcctacaagcacatttctattgcatacagactttgagcaggttatgtgtattaaagacaataaaattgttaaattgttactttgtcaaaactatgaccccctattagtggggcagcatgtgttaaagtggattgataaaaaatgtggcaagaaaaatacactgtggttttatgggccgccaagtacaggaaaaacaaacttggcaatggccattgctaaaagtgttccagtatatggcatggttaactggaataatgaaaactttccatttaatgatgtagcagggaaaagcttggtggtctgggatgaaggtattattaagtctacaattgtagaagctgcaaaagccattttaggcgggcaacccaccagggtagatcaaaaaatgcgtggaagtgtagctgtgcctggagtacctgtggttataaccagcaatggtgacattacttttgttgtaagcgggaacactacaacaactgtacatgctaaagccttaaaagagcgaatggtaaagttaaactttactgtaagatgcagccctgacatggggttactaacagaggctgatgtacaacagtggcttacatggtgtaatgcacaaagctgggaccactatgaaaactgggcaataaactacacttttgatttccctggaattaatgcagatgccctccacccagacctccaaaccaccccaattgtcacagacaccagtatcagcagcagtggtggtgaaagctctgaagaactcagtgaaagcagcttttttaacctcatcaccccaggcgcctggaacactgaaaccccgcgctctagtacgcccatccccgggaccagttcaggagaatcatttgtcggaagctcagtttcctccgaagttgtagctgcatcgtgggaagaagccttctacacacctttggcagaccagtttcgtgaactgttagttggggttgattatgtgtgggacggtgtaaggggtttacctgtgtgttgtgtgcaacatattaacaatagtgggggaggcttgggactttgtccccattgcattaatgtaggggcttggtataatggatggaaatttcgagaatttaccccagatttggtgcggtgtagctgccatgtgggagcttctaatcccttttctgtgctaacctgcaaaaaatgtgcttacctgtctggattgcaaagctttgtagattatgagtaaagaaagtggcaaatggtgggaaagtgatgataaatttgctaaagctgtgtatcagcaatttgtggaattttatgaaaaggttactggaacagacttagagcttattcaaatattaaaagatcactataatatttctttagataatcccctagaaaacccatcctctctgtttgacttagttgctcgtattaaaaataaccttaaaaactctccagacttatatagtcatcattttcaaagtcatggacagttatctgaccacccccatgccttatcatccagtagcagtcatgcagaacctagaggagaaaatgcagtattatctagtgaagacttacacaagcctgggcaagttagcgtacaactacccggtactaactatgttgggcctggcaatgagctacaagctgggcccccgcaaagtgctgttgacagtgctgcaaggattcatgactttaggtatagccaactggctaagttgggaataaatccatatactcattggactgtagcagatgaagagcttttaaaaaatataaaaaatgaaactgggtttcaagcacaagtagtaaaagactactttactttaaaaggtgcagctgcccctgtggcccattttcaaggaagtttgccggaagttcccgcttacaacgcctcagaaaaatacccaagcatgacttcagttaattctgcagaagccagcactggtgcaggagggggtggcagtaatcctgtcaaaagcatgtggagtgagggggccacttttagtgccaactctgtaacttgtacattttccagacagtttttaattccttatgacccagagcaccattataaggtgttttctcccgcagcaagcagctgccacaatgccagtggaaaggaggcaaaggtttgcacaattagtcccataatgggatactcaaccccatggagatatttagattttaatgctttaaatttatttttttcacctttagagtttcagcacttaattgaaaattatggaagtatagctcctgatgctttaactgtaaccatatcagaaattgctgttaaggatgttacagacaaaactggagggggggtacaggttactgacagcactacagggcgcctatccatgttagtagaccatgaatacaagtacccatatgtgttaggacaaggtcaggatactttagccccagaacttcctatttgggtatactttccccctcaatatgcttacttaacagtaggagatgttaacacacaaggaatctctggagacagcaaaaaattagcaagtgaagaatcagcattttatgttttggaacacagttcttttcagcttttaggtacaggaggtacagcaactatgtcttataagtttcctccagtgcccccagaaaatttagagggctgcagtcaacacttttatgaaatgtacaatcccttatacggatcccgcttaggggttcctgacacattaggaggtgacccaaaatttagatctttaacacatgaagaccatgcaattcagccccaaaacttcatgccagggccactagtaaactcagtgtctacaaaggagggagacagctctaatactggagctggaaaagccttaacaggccttagcacaggcacctctcaaaacactagaatatccttacgccctgggccagtgtcacagccataccaccactgggacacagataaatatgttccaggaataaatgccatttctcatggtcagaccacttatggtaacgctgaagacaaagagtatcagcaaggagtgggtagatttccaaatgaaaaagaacagctaaaacagttacagggtttaaacatgcacacctatttccccaataaaggaacccagcaatatacagatcaaattgagcgccccctaatggtgggttctgtatggaacagaagagcccttcactatgaaagccagctgtggagtaaaattccaaatttagatgacagttttaaaactcagtttgcagccttaggaggatggggtttgcatcagccacctcctcaaatatttttaaaaatattaccacaaagtgggccaattggaggtattaaatcaatgggaattactaccttagttcagtatgccgtgggaattatgacagtaactatgacatttaaattggggccccgtaaagctacgggacggtggaatcctcaacctggagtatatcccccgcacgcagcaggtcatttaccatatgtactatatgaccccacagctacagatgcaaaacaacaccacaggcatggatacgaaaagcctgaagaattgtggacagccaaaagccgtgtgcacccattgtaaacactccccaccgtgccctcagccaggatgcgtaactaaacgcccaccagtaccacccagactgtacctgccccctcctgtacctataagacagcctaacacaaaagatatagacaatgtagaatttaagtacttaaccagatatgaacaacatgttattagaatgttaagattgtgtaatatgtatcaaaatttagaaaaataaacatttgttgtggttaaaaaattatgttgttgcgctttaaaaatttaaaagaagacaccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcgcttggggttgacgtgccactaagacaagcggcgcgccgcttgtcttagtgtcaaggcaaccccaagcaagctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttgg

SEQ ID NO: 2 - DNA, 3221 bp, Hepatitis B virus
ttccactgccttccaccaagctctgcaagaccccagagtcaggggtctgtattttcctgctggtggctccagttcaggaacagtaaaccctgctccgaatattgcctctcacatctcgtcaatctccgcgaggaccggggaccctgtgacgaacatggagaacatcacatcaggattcctaggacccctgcccgtgttacaggcggggtttttcttgttgacaagaatcctcacaataccgcagagtctagactcgtggtggacttctctcaattttctagggggatcacccgtgtgtcttggccaaaattcgcgatccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattctaggatcaacaacaaccagtacgggaccatgcaaaacctgcacgactcctgctcaaggcaactctatgtttccctcatgttgctgtacaaaacctacggatggaaattgcacctgtattcccatcccatcgtcttgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagttcctttataccgctgttaccaattttcttttgtctctgggtatacatttaaaccctaacaaaacaaaaagatggggttattccctaaacttcatgggttatgtaattggaagttggggaacattgccacaggatcatattgtacaaaaaatcaaacactgttttagaaaacttcctgttaacaggcctattgattggaaagtatgtcaaagaattgtgggtcttttgggctttgctgctccttttacacaatgtggatatcctgccttaatgcccttgtatgcatgtatacaagctaaacaggctttcactttctcgccaacttacaaggcctttctaagtaaacagtacatgaacctttaccccgttgctcggcaacggcctggtctgtgccaagtatttgctgatgcaacccccactggctggggcttggccataggccatcagcgcatgcgcggaacctttgtggctcctctgccgatccatactgcggaactcctagccgcttgttttgctcgcagccggtctggagcgaaactcatcggaactgacaattctgtcgtcctctcgcggaaatatacctcgtttccatggctactaggctgtgctgccaactggatccttcgcgggacgtcctttgtttacgtcccgtcggcgctgaatcccgcggacgacccctctcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttctttccttccgtcagagatctcctagacaccgcctcggctctgtatcgggaagccttagagtctcctgagcattgctcacctcaccataccgcactcaggcaagccattctctgctggggggaattgatgactctagctacctgggtgggtaataatttggaagatccagcatccagggatctagtagtcaattatgttaatactaacatgggattaaagatcaggcaactcttgtggtttcatatctcttgccttacttttggaagagaaactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgttagtattccttggactcataaggtgggaaacttcactgggctttattcctctacagcacctatctttaatcctgaatggcaaactccttcctttcctaaaattcatttacaagaggacattattaataggtgtcaacaatttgtgggccctctcactgtaaatgaaaagagaagattgaaattaattatgcctgctagattctatcctacccacactaaatatttgcccttagacaaaggaattaaaccttattatccagatcaggtagttaatcattacttccaaaccagacattatttacatactctttggaaggcgggtattctatataagagagaaaccacacgtagcgcatcattttgcgggtcaccatattcttgggaacaagagctacagcatgggaggttggtcatcaaaacctcgcaaaggcatggggacgaatctttctgttcccaaccctctgggattctttcccgatcatcagttggaccctgtattcggagccaactcaaacaatccagattgggacttcaaccccatcaaggaccactggccagcagccaaccaggtaggagtgggagcattcgggccagggttcacccctccacacggcggtgttttggggtggagccctcaggctcagggcatgttgaccccagtgtcaacaattcctcctcctgcctccgccaatcggcagtcaggaaggcagcctactcccatctctccacctctaagagacagtcatcctcaggccatgcagtggaa

SEQ ID NO: 3 - DNA, 5075 bp, Porcine parvovirus
aatctttaaactgaccaactgtctttgcgtatggtgacgtgatgacgcgcgctacgcgcgctgccttcggcagtcacacgtcaccatcagcaaagacagttggtcagtttaaagattaataagacattccattggctgaaaagaggcgggaaattcaaaaaaagaggcgggaaaaaaagaggtggagcctaacactataaatacagttgcttacttcagttagttcctttctgcttcagactgcacttcgctccagagacacagctacaaactactctcagctactgcagcatggcagcgggaaacacttactcggaagaggtactaaaagctaccaactggcttcaagataatgctcaaaaagaagcattctcttatgtatttaaaacacaaaaagtcaatctaaatggaaaagaaattgcttggaataactacaacaaagatacaacagatgcggaaatgataaacctacaaagaggagcagaaacatcatgggaccaggcaacagacatggaatgggaatcagaaatcgacagcctcacaaaacggcaagtactgatttttgactctcttgttaaaaaatgtctctttgaaggtatattgcaaaagaacctaagtccaagtgactgctactggttcatacagcatgaacatggtcaagatactggctatcactgccatgtactactaggtggaaaaggcttacaacaagcaatgggaaaatggttcagaaaacaattaaacaatttatggagtagatggttaataatgcaatgcaaagtacctctaacaccagttgaaagaataaaattaagggaattagcagaggatggtgagtgggtatcgctactaacctacactcacaaacaaactaaaaaacaatatacaaaaatgactcattttggaaatatgattgcttactacttcctaaataaaaaaagaaagacaactgaaagagagcatggatattatctcagctcagattctggcttcatgacaaatttcttaaaagaaggcgagagacacttagtcagtcacctatttactgaagcaaataaacctgaaactgtggaaacaacggttactacagctcaggaagccaaaagaggcagaatacaaacaaaaaaagaagtaagcataaaatgcacaataagagacttggttaataaaagatgtactagcatagaagactggatgatgacagatccagacagttatatagaaatgatggctcaaaccggaggagaaaatttaatcaaaaatacactagaaataacaactcttactctagcaagaacaaaaacagcatatgacttaatacttgaaaaggcaaaaccaagcatgctaccaacatttaatattagcaatacaagaacatgtaaaatattcagcatgcacaattggaactacattaaagtctgccatgctataacttgtgtactaaacagacaaggaggaaaaagaaatacaattctatttcatgggccagcatcaacaggaaaaagtataattgctcaacacattgcaaacttagttggtaatgttggttgctacaatgcagccaatgtgaactttccatttaatgactgtacaaataaaaacttaatatggattgaagaagcaggaaacttctctaaccaagtaaaccaattcaaagccatatgttcaggtcaaacaattagaattgaccaaaaaggtaaaggaagcaaacaaattgaaccaactcctgtaataatgactacaaatgaagacataactaaagttagaataggatgcgaggaaagaccagaacatacacaaccaataagagacagaatgttaaacataaacctaaccagaaaactgccaggtgattttggacttttagaagaaactgaatggccactaatatgtgcttggttggtaaagaaaggttaccaagcaacaatggctagctatatgcatcattggggaaatgtacctgattggtcagaaaaatgggaggagccaaaaatgcaaaccccaataaatacaccaacagactctcagatttccacatcagtgaaaacttcgccagcggacaacaactacgcagcaactccaatacaggaggacctggatttagctttagccttggagccgtggagcgagccaacaacaccaactttcaccaacctgcacttaactccaacaccgccagattcagcaatacggacaccaagtccaacttggtcggaaatagaaaccgacataagagcctgctttggtgaaaactgtgcacccacaacaaaccttgaataaggtaggatggcgcctcctgcaaaaagagcaagaggtaagggtagttttaagggggtggtgggcatacatataaaactaactgcaaataatttttttatatattacaggactaactctaccaggatacaaataccttggtccaggaaactcactagaccaaggagaaccaactaatccatcagacgccgcagcaaaagaacacgacgaagcctacgacaaatacataaaatctggaaaaaatccatacttctacttctcagcagctgatgaaaaattcataaaagaaactgaacacgcaaaagactacggaggtaaaattggacattacttcttcagagcaaagcgtgcctttgctccaaaactctcagaaacagactcaccaactacatctcaacaaccagaggtaagaagatcgccgagaaaacacccagggtctaaaccaccaggaaaaagacctgctccaagacatatttttataaacttagctaaaaaaaaagctaaagggacatctaatacaaactctaactcaatgagtgaaaatgtggaacaacacaaccctattaatgcaggcactgaattgtctgcaacaggaaatgaatctgggggtgggggcggcggtggcgggggtaggggtgctgggggggttggtgtgtctacaggtactttcaataatcaaacagaatttcaatacttgggggagggcttggttagaatcactgcacacgcatcaagactcatacatctaaatatgccagaacacgaaacatacaaaagaatacatgtactaaattcagaatcaggggtggcgggacaaatggtacaagacgatgcacacacacaaatggtaacaccttggtcactaatagatgctaacgcatggggagtgtggttcaatccagcggactggcagttaatatccaacaacatgacagaaataaacttagttagttttgaacaagaaatattcaatgtagtacttaaaacaattacagaatcagcaacctcaccaccaaccaaaatatataataatgatctaactgcaagcttaatggtcgcactagacaccaataacacacttccatacacaccagcagcacctagaagtgaaacacttggtttttatccatggttacctacaaaaccaactcaatacagatattacctatcatgcatcagaaacctaaatccaccaacatacactggacaatcacaacaaataacagactcaatacaaacaggactacacagtgacattatgttctacacaatagaaaatgcagtaccaattcatcttctaagaacaggagatgaattctccacaggaatatatcactttgacacaaaaccactaaaattaactcactcatggcaaacaaacagatctctaggactgcctccaaaactactaactgaacctaccacagaaggagaccaacacccaggaacactaccagcagctaacacaagaaaaggttatcaccaaacaattaataatagctacacagaagcaacagcaattaggccagctcaggtaggatataatacaccatacatgaattttgaatactccaatggtggaccatttctaactcctatagtaccaacagcagacacacaatataatgatgatgaaccaaatggtgctataagatttacaatggattaccaacatggacacttaaccacatcttcacaagagctagaaagatacacattcaatccacaaagtaaatgtggaagagctccaaagcaacaatttaatcaacaggcaccactaaacctagaaaatacaaataatggaacacttttaccttcagatccaataggagggaaatctaacatgcatttcatgaatacactcaatacatatggaccattaacagcactaaacaatactgcacctgtatttccaaatggtcaaatatgggataaagaacttgatacagatctaaaacctagactacatgttacagctccatttgtttgtaaaaacaatccaccaggacaactatttgtaaaaatagcaccaaacctaacagatgatttcaatgctgactctcctcaacaacctagaataataacttattcaaacttttggtggaaaggaacactaacattcacagcaaaaatgagatccagtaatatgtggaaccctattcaacaacacacaacaacagcagaaaacattggtaactatattcctacaaatattggtggcataagaatgtttccagaatattcacaacttataccaagaaaattatactagaaataactctgtaaataaaaactcagttacttggttaatcatgtactactatcattgtatacttcaataaaaataaattgtaaaatcaataaaactaagttacttagtttctgtatacctatactagaaataactctgtaaataaaaactcagttacttggttaatcatgtactactatcattgtatacttcaataaaaataaattgtaaaatcaataaaactaagttacttagtttctgtataccaattatccccaaaaaacaataaaattttaaaaagaaacaagctctcatgtgtttactattaactaaaccaaccacacttatatgaccttatgtctttagggtgggtgggtgggaattactatgtattcctttgagttagttggtcgcctttgggcgactaaccaagcggctctgccgcttggttagtcgcacggcgaccaactaactcaaaggaatacatagtaattcccacccacccaccctaaagacataaggtcatataagtgtggttggtttagtt

SEQ ID NO: 4 - DNA, 11703 bp, Sindbis virus
attgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccatgaatagaggattctttaacatgctcggccgccgccccttcccggcccccactgccatgtggaggccgcggagaaggaggcaggcggccccgatgcctgcccgcaacgggctggcttctcaaatccagcaactgaccacagccgtcagtgccctagtcattggacaggcaactagacctcaacccccacgtccacgcccgccaccgcgccagaagaagcaggcgcccaagcaaccaccgaagccgaagaaaccaaaaacgcaggagaagaagaagaagcaacctgcaaaacccaaacccggaaagagacagcgcatggcacttaagttggaggccgacagattgttcgacgtcaagaacgaggacggagatgtcatcgggcacgcactggccatggaaggaaaggtaatgaaacctctgcacgtgaaaggaaccatcgaccaccctgtgctatcaaagctcaaatttaccaagtcgtcagcatacgacatggagttcgcacagttgccagtcaacatgagaagtgaggcattcacctacaccagtgaacaccccgaaggattctataactggcaccacggagcggtgcagtatagtggaggtagatttaccatccctcgcggagtaggaggcagaggagacagcggtcgtccgatcatggataactccggtcgggttgtcgcgatagtcctcggtggcgctgatgaaggaacacgaactgccctttcggtcgtcacctggaatagtaaagggaagacaattaagacgaccccggaagggacagaagagtggtccgcagcaccactggtcacggcaatgtgtttgctcggaaatgtgagcttcccatgcgaccgcccgcccacatgctatacccgcgaaccttccagagccctcgacatccttgaagagaacgtgaaccatgaggcctacgataccctgctcaatgccatattgcggtgcggatcgtctggcagaagcaaaagaagcgtcattgacgactttaccctgaccagcccctacttgggcacatgctcgtactgccaccatactgtaccgtgcttcagccctgttaagatcgagcaggtctgggacgaagcggacgataacaccatacgcatacagacttccgcccagtttggatacgaccaaagcggagcagcaagcgcaaacaagtaccgctacatgtcgcttaagcaggatcacaccgttaaagaaggcaccatggatgacatcaagattagcacctcaggaccgtgtagaaggcttagctacaaaggatactttctcctcgcaaaatgccctccaggggacagcgtaacggttagcatagtgagtagcaactcagcaacgtcatgtacactggcccgcaagataaaaccaaaattcgtgggacgggaaaaatatgatctacctcccgttcacggtaaaaaaattccttgcacagtgtacgaccgtctgaaagaaacaactgcaggctacatcactatgcacaggccgagaccgcacgcttatacatcctacctggaagaatcatcagggaaagtttacgcaaagccgccatctgggaagaacattacgtatgagtgcaagtgcggcgactacaagaccggaaccgtttcgacccgcaccgaaatcactggttgcaccgccatcaagcagtgcgtcgcctataagagcgaccaaacgaagtgggtcttcaactcaccggacttgatcagacatgacgaccacacggcccaagggaaattgcatttgcctttcaagttgatcccgagtacctgcatggtccctgttgcccacgcgccgaatgtaatacatggctttaaacacatcagcctccaattagatacagaccacttgacattgctcaccaccaggagactaggggcaaacccggaaccaaccactgaatggatcgtcggaaagacggtcagaaacttcaccgtcgaccgagatggcctggaatacatatggggaaatcatgagccagtgagggtctatgcccaagagtcagcaccaggagaccctcacggatggccacacgaaatagtacagcattactaccatcgccatcctgtgtacaccatcttagccgtcgcatcagctaccgtggcgatgatgattggcgtaactgttgcagtgttatgtgcctgtaaagcgcgccgtgagtgcctgacgccatacgccctggccccaaacgccgtaatcccaacttcgctggcactcttgtgctgcgttaggtcggccaatgctgaaacgttcaccgagaccatgagttacttgtggtcgaacagtcagccgttcttctgggtccagttgtgcatacctttggccgctttcatcgttctaatgcgctgctgctcctgctgcctgccttttttagtggttgccggcgcctacctggcgaaggtagacgcctacgaacatgcgaccactgttccaaatgtgccacagataccgtataaggcacttgttgaaagggcagggtatgccccgctcaatttggagatcactgtcatgtcctcggaggttttgccttccaccaaccaagagtacattacctgcaaattcaccactgtggtcccctccccaaaaatcaaatgctgcggctccttggaatgtcagccggccgctcatgcagactatacctgcaaggtcttcggaggggtctacccctttatgtggggaggagcgcaatgtttttgcgacagtgagaacagccagatgagtgaggcgtacgtcgaattgtcagcagattgcgcgtctgaccacgcgcaggcgattaaggtgcacactgccgcgatgaaagtaggactgcgtattgtgtacgggaacactaccagtttcctagatgtgtacgtgaacggagtcacaccaggaacgtctaaagacttgaaagtcatagctggaccaatttcagcatcgtttacgccattcgatcataaggtcgttatccatcgcggcctggtgtacaactatgacttcccggaatatggagcgatgaaaccaggagcgtttggagacattcaagctacctccttgactagcaaggatctcatcgccagcacagacattaggctactcaagccttccgccaagaacgtgcatgtcccgtacacgcaggcctcatcaggatttgagatgtggaaaaacaactcaggccgcccactgcaggaaaccgcacctttcgggtgtaagattgcagtaaatccgctccgagcggtggactgttcatacgggaacattcccatttctattgacatcccgaacgctgcctttatcaggacatcagatgcaccactggtctcaacagtcaaatgtgaagtcagtgagtgcacttattcagcagacttcggcgggatggccaccctgcagtatgtatccgaccgcgaaggtcaatgccccgtacattcgcattcgagcacagcaactctccaagagtcgacagtacatgtcctggagaaaggagcggtgacagtacactttagcaccgcgagtccacaggcgaactttatcgtatcgctgtgtgggaagaagacaacatgcaatgcagaatgtaaaccaccagctgaccatatcgtgagcaccccgcacaaaaatgaccaagaatttcaagccgccatctcaaaaacatcatggagttggctgtttgcccttttcggcggcgcctcgtcgctattaattataggacttatgatttttgcttgcagcatgatgctgactagcacacgaagatgaccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttc

SEQ ID NO: 5 - DNA, 10945 bp, West Nile virus
gaggattaacaacaattaacacagtgcgagctgtttcttagcacgaagatctcgatgtctaagaaaccaggagggcccggcaagagccgggctgtcaatatgctaaaacgcggaatgccccgcgtgttgtccttgattggactgaagagggctatgttgagcctgatcgacggcaaggggccaatacgatttgtgttggctctcttggcgttcttcaggttcacagcaattgctccgacccgagcagtgctggatcgatggagaggtgtgaacaaacaaacagcgatgaaacaccttctgagttttaagaaggaactagggaccttgaccagtgctatcaatcggcggagctcaaaacaaaagaaaagaggaggaaagaccggaattgcagtcatgattggcctgatcgccagcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacctatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagcttcaactgccttggaatgagcaacagagacttcttggaaggagtgtctggagcaacatgggtggatttggttctcgaaggcgatagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccgaccatgggggaagcccacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccatcttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgataggtccatagctctcacgtttctcgcagttggaggagttctgctcttcctctccgtgaacgtgcacgctgacactgggtgtgccataaacatcacccggcaagagctgagatgtggaagtggagtgttcatacacaatgatgtggaggcttggatggaccggtacaagtattaccctgaaacgccacaaggcctagccaagatcattcagaaagctcataaggaaggagtgtgcggtctacgatcagtttccagactggagcatcaaatgtgggaagcagtgaaggacgagctgaacactcctttgaaggagaatggtgtggaccttagtgtcgtggttgagaaacaggagggaatgtacaagtcagcacctaaacgcctcaccgccaccacggaaaaattggaaattggctggaaggcctggggaaagagtattttatttgcaccagaactcgccaacaacacctttgtggttgatggtccggagaccaaggaatgtccgactcagaatcgcgcttggaatagcttagaagtggaggattttggatttggtctcaccagcactcggatgttcctgaaggtcagagaaggcaacacaactgaatgtgactcgaagatcattggaacggctgtcaagaacaacttggcgatccacagtgacctgtcctattggattgaaagcaggctcaatgatacgtggaagcttgaaagggcagttctgggtgaagtcaaatcatgtacgtggcctgagacgcataccttgtggggcgatggaatccttgagagtgacttgataataccagtcacactggcgggaccacgaagcaatcacaatcggagacctgggtacaagacacaaaaccagggcccatgggacgaaggccgggtagagattgacttcgattactgcccaggaactacggtcaccctgagtgagagctgcggacaccgtggacctgccactcgcaccaccacagagagcggaaagttgataacagattggtgctgcaggagctgcaccttaccaccactgcgctaccaaactgacagcggctgttggtatggtatggagatcagaccacagagacatgatgaaaagaccctcgtgcagtcacaagtgaatgcttataatgctgatatgattgacccttttcagttgggccttctggtcgtgttcttggccacccaggaggtccttcgcaagaggtggacagccaagatcagcatgccagctatactgattgctctgctagtcctggtgtttgggggcattacttacactgatgtgttacgctatgtcatcttggtgggggcagctttcgcagaatctaattcgggaggagacgtggtacacttggcgctcatggcgaccttcaagatacaaccagtgtttatggtggcatcgtttctcaaagcgagatggaccaaccaggagaacattttgttgatgttggcggctgttttctttcaaatggcttatcacgatgcccgccaaattctgctctgggagatccctgatgtgttgaattcactggcggtagcttggatgatactgagagccataacattcacaacgacatcaaacgtggttgttccgctgctagccctgctaacacccgggctgagatgcttgaatctggatgtgtacaggatactgctgttgatggtcggaataggcagcttgatcagggagaagaggagtgcagctgcaaaaaagaaaggagcaagtctgctatgcttggctctagcctcaacaggacttttcaaccccatgatccttgctgctggactgattgcatgtgatcccaaccgtaaacgcggatggcccgcaactgaagtgatgacagctgtcggcctaatgtttgccatcgtcggagggctggcagagcttgacattgactccatggccattccaatgactatcgcggggctcatgtttgctgctttcgtgatttctgggaaatcaacagatatgtggattgagagaacggcggacatttcctgggaaagtgatgcagaaattacaggctcgagcgaaagagttgatgtgcggcttgatgatgatggaaacttccagctcatgaatgatccaggagcaccttggaagatatggatgctcagaatggtctgtctcgcgattagtgcgtacaccccctgggcaatcttgccctcagtagttggattttggataactctccaatacacaaagagaggaggcgtgttgtgggacactccctcaccaaaggagtacaaaaagggggacaccaccaccggcgtttacaggatcatgactcgtgggctgctcggcagttatcaagcaggagcgggcgtgatggttgaaggtgttttccacaccctttggcatacaacaaaaggagccgctttgatgagcggagagggccgcctggacccatactggggcagtgtcaaggaggatcgactttgttacggaggaccctggaaattgcagcacaagtggaacgggcaggatgaggtgcagatgattgtggtggaacctggcaagaacgttaagaacgtccagacgaaaccaggggtgttcaaaacacctgaaggagaaatcggggccgtgactttggacttccccactggaacatcaggctcaccaatagtggacaaaaacggtgatgtgattgggctttatggcaatggagtcataatgcccaacggctcatacataagcgcgatagtgcagggtgaaaggatggatgagccaatcccagccggattcgaacctgagatgctgaggaaaaaacagatcactgtactggatctccatcccggcgccggtaaaacaaggaggattctgccacagatcatcaaagaggccataaacagaagactgagaacagccgtgctagcaccaaccagggttgtggctgctgagatggctgaagcactgagaggactgcccatccggtaccagacatccgcagtgcccagagaacataatggaaatgagattgttgatgtcatgtgtcatgctaccctcacccacaggctgatgtctcctcacagggtgccgaactacaacctgttcgtgatggatgaggctcatttcaccgacccagctagcattgcagcaagaggttacatttccacaaaggtcgagctaggggaggcggcggcaatattcatgacagccaccccaccaggcacttcagatccattcccagagtccaattcaccaatttccgacttacagactgagatcccggatcgagcttggaactctggatacgaatggatcacagaatacaccgggaagacggtttggtttgtgcctagtgtcaagatggggaatgagattgccctttgcctacaacgtgctggaaagaaagtagtccaattgaacagaaagtcgtacgagacggagtacccaaaatgtaagaacgatgattgggactttgttatcacaacagacatatctgaaatgggggctaacttcaaggcgagcagggtgattgacagccggaagagtgtgaaaccaaccatcataacagaaggagaagggagagtgatcctgggagaaccatctgcagtgacagcagctagtgccgcccagagacgtggacgtatcggtagaaatccgtcgcaagttggtgatgagtactgttatggggggcacacgaatgaagacgactcgaacttcgcccattggactgaggcacgaatcatgctggacaacatcaacatgccaaacggactgatcgctcaattctaccaaccagagcgtgagaaggtatataccatggatggggaataccggctcagaggagaagagagaaaaaactttctggaactgttgaggactgcagatctgccagtttggctggcttacaaggttgcagcggctggagtgtcataccacgaccggaggtggtgctttgatggtcctaggacaaacacaattttagaagacaacaacgaagtggaagtcatcacgaagcttggtgaaaggaagattctgaggccgcgctggattgacgccagggtgtactcggatcaccaggcactaaaggcgttcaaggacttcgcctcgggaaaacgttctcagatagggctcattgaggttctgggaaagatgcctgagcacttcatggggaagacatgggaagcacttgacaccatgtacgttgtggccactgcagagaaaggaggaagagctcacagaatggccctggaggaactgccagatgctcttcagacaattgccttgattgccttattgagtgtgatgaccatgggagtattcttcctcctcatgcagcggaagggcattggaaagataggtttgggaggcgctgtcttgggagtagcgacctttttctgttggatggctgaagttccaggaacgaagatcgccggaatgttgctgctctcccttctcttgatgattgtgctaattcctgagccagagaagcaacgttcgcagacagacaaccagctagccgtgttcctgatttgtgtcatgacccttgtgagcgcagtggcagccaacgagatgggttggctagataagaccaagagtgacataagcagtttgtttgggcaaagaattgaggtcaaggagaatttcagcatgggagagtttcttctggacttgaggccggcaacagcctggtcactgtacgctgtgacaacagcggtcctcactccactgctaaagcatttgatcacgtcagattacatcaacacctcattgacctcaataaacgttcaggcaagtgcactattcacactcgcgcgaggcttccccttcgtcgatgttggagtgtcggctctcctgctagcagccggatgctggggacaagtcaccctcaccgttacggtaacagcggcaacactccttttttgccactatgcctacatggttcccggttggcaagctgaggcaatgcgctcagcccagcggcggacagcggccggaatcatgaagaacgctgtagtggatggcatcgtggccacggacgtcccagaattagagcgcaccacacccatcatgcagaagaaagttggacagatcatgctgatcttggtgtctctagctgcagtagtagtgaacccgtctgtgaagacagtacgagaagccggaattttgatcacggccgcagcggtgacgctttgggagaatggagcaagctctgtttggaacgcaacaactgccatcggactctgccacatcatgcgtgggggttggttgtcatgtctatccataacatggacactcataaagaacatggaaaaaccagggctaaaaagaggtggggcaaaaggacgcaccttgggagaggtttggaaagaaagactcaaccagatgacaaaagaagagttcactaggtaccgcaaagaggccatcatcgaagtcgatcgctcagcggcaaaacacgccaggaaagaaggcaatgtcactggagggcatccagtctctaggggcacagcaaaactgagatggctggtcgaacggaggtttctcgaaccggtcggaaaagtgattgaccttggatgtggaagaggcggttggtgttactatatggcaacccaaaaaagagtccaagaagtcagagggtacacaaagggcggtcccggacatgaagagccccaactagtgcaaagttatggatggaacattgtcaccatgaagagtggagtggatgtgttctacagaccttctgagtgttgtgacaccctcctttgtgacatcggagagtcctcgtcaagtgctgaggttgaagagcataggacgattcgggtccttgaaatggttgaggactggctgcaccgagggccaagggaattttgcgtgaaggtgctctgcccctacatgccgaaagtcatagagaagatggagctgctccaacgccggtatggggggggactggtcagaaacccactctcacggaattccacgcacgagatgtattgggtgagtcgagcttcaggcaatgtggtacattcagtgaatatgaccagccaggtgctcctaggaagaatggaaaaaaggacctggaagggaccccaatacgaggaagatgtaaacttgggaagtggaaccagggcggtgggaaaacccctgctcaactcagacaccagtaaaatcaagaacaggattgaacgactcaggcgtgagtacagttcgacgtggcaccacgatgagaaccacccatatagaacctggaactaccacggcagttatgatgtgaagcccacaggctccgccagttcgctggtcaatggagtggtcaggctcctctcaaaaccatgggacaccatcacgaatgttaccaccatggccatgactgacactactcccttcgggcagcagcgagtgttcaaagagaaggtggacacgaaagctcctgaaccgccagaaggagtgaagtacgtgctcaacgagaccaccaactggttgtgggcgtttttggccagagaaaaacgtcccagaatgtgctctcgagaggaattcataagaaaggtcaacagcaatgcagctttgggtgccatgtttgaagagcagaatcaatggaggagcgccagagaagcagttgaagatccaaaattttgggagatggtggatgaggagcgcgaggcacatctgcggggggaatgtcacacttgcatttacaacatgatgggaaagagagagaaaaaacccggagagttcggaaaggccaagggaagcagagccatttggttcatgtggctcggagctcgctttctggagttcgaggctctgggttttctcaatgaagaccactggcttggaagaaagaactcaggaggaggtgtcgagggcttgggcctccaaaaactgggttacatcctgcgtgaagttggcacccggcctgggggcaagatctatgctgatgacacagctggctgggacacccgcatcacgagagctgacttggaaaatgaagctaaggtgcttgagctgcttgatggggaacatcggcgtcttgccagggccatcattgagctcacctatcgtcacaaagttgtgaaagtgatgcgcccggctgctgatggaagaaccgtcatggatgttatctccagagaagatcagagggggagtggacaagttgtcacctacgccctaaacactttcaccaacctggccgtccagctggtgaggatgatggaaggggaaggagtgattggcccagatgatgtggagaaactcacaaaagggaaaggacccaaagtcaggacctggctgtttgagaatggggaagaaagactcagccgcatggctgtcagtggagatgactgtgtggtaaagcccctggacgatcgctttgccacctcgctccacttcctcaatgctatgtcaaaggttcgcaaagacatccaagagtggaaaccgtcaactggatggtatgattggcagcaggttccattttgctcaaaccatttcactgaattgatcatgaaagatggaagaacactggtggttccatgccgaggacaggatgaattggtaggcagagctcgcatatctccaggggccggatggaacgtccgcgacactgcttgtctggctaagtcttatgcccagatgtggctgcttctgtacttccacagaagagacctgcggctcatggccaacgccatttgctccgctgtccctgtgaattgggtccctaccggaagaaccacgtggtccatccatgcaggaggagagtggatgacaacagaggacatgttggaggtctggaaccgtgtttggatagaggagaatgaatggatggaagacaaaaccccagtggagaaatggagtgacgtcccatattcaggaaaacgagaggacatctggtgtggcagcctgattggcacaagagcccgagccacgtgggcagaaaacatccaggtggctatcaaccaagtcagagcaatcatcggagatgagaagtatgtggattacatgagttcactaaagagatatgaagacacaactttggttgaggacacagtactgtagatatttaatcaattgtaaatagacaatataagtatgcataaaagtgtagttttatagtagtatttagtggtgttagtgtaaatagttaagaaaattttgaggagaaagtcaggccgggaagttcccgccaccggaagttgagtagacggtgctgcctgcgactcaaccccaggaggactgggtgaacaaagccgcgaagtgatccatgtaagccctcagaaccgtctcggaaggaggaccccacatgttgtaacttcaaagcccaatgtcagaccacgctacggcgtgctactctgcggagagtgcagtctgcgatagtgccccaggaggactgggttaacaaaggcaaaccaacgccccacgcggccctagccccggtaatggtgttaaccagggcgaaaggactagaggttagaggagaccccgcggtttaaagtgcacggcccagcctggctgaagctgtaggtcaggggaaggactagaggttagtggagaccccgtgccacaaaacaccacaacaaaacagcatattgacacctgggatagactaggagatcttctgctctgcacaaccagccacacggcacag

SEQ ID NO: 6 - DNA, 1542 bp, Escherichia coli 16S Ribosomal RNA
  misc_feature (896)..(896): n is a, c, g, or t
aaattgaagagtttgatcatggctcagattgaacgctggcggcaggcctaacacatgcaagtcgaacggtaacaggaakcagcttgctgatttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtckaccgcctggggagtacggncgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgayctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 7 - DNA, 2905 bp, Escherichia coli 23 S Ribosomal RNA
ggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaagcgacttgctcgtggagctgaaatcagtcgaagataccagctggctgcaactgtttattaaaaacacagcactgtgcaaacacgaaagtggacgtatacggtgtgacgcctgcccggtgccggaaggttaattgatggggttagcggtaacgcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttggcccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatgcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaacctt

SEQ ID NO: 8 - DNA, 1798 bp, Yeast (S. cerevisiae)
tatctggttgatcctgccagtagtcatatgcttgtctcaaagattaagccatgcatgtctaagtataagcaatttatacagtgaaactgcgaatggctcattaaatcagttatcgtttatttgatagttcctttactacatggtataaccgtggtaattctagagctaatacatgcttaaaatctcgaccctttggaagagatgtatttattagataaaaaatcaatgtcttcggactctttgatgattcataataacttttcgaatcgcatggccttgtgctggcgatggttcattcaaatttctgccctatcaactttcgatggtaggatagtggcctaccatggtttcaacgggtaacggggaataagggttcgattccggagagggagcctgagaaacggctaccacatccaaggaaggcagcaggcgcgcaaattacccaatcctaattcagggaggtagtgacaataaataacgatacagggcccattcgggtcttgtaattggaatgagtacaatgtaaataccttaacgaggaacaattggagggcaagtctggtgccagcagccgcggtaattccagctccaatagcgtatattaaagttgttgcagttaaaaagctcgtagttgaactttgggcccggttggccggtccgattttttcgtgtactggatttccaacggggcctttccttctggctaaccttgagtccttgtggctcttggcgaaccaggacttttactttgaaaaaattagagtgttcaaagcaggcgtattgctcgaatatattagcatggaataatagaataggacgtttggttctattttgttggtttctaggaccatcgtaatgattaatagggacggtcgggggcatcggtattcaattgtcgaggtgaaattcttggatttattgaagactaactactgcgaaagcatttgccaaggacgttttcattaatcaagaacgaaagttaggggatcgaagatgatctggtaccgtcgtagtcttaaccataaactatgccgactagatcgggtggtgtttttttaatgacccactcggtaccttacgagaaatcaaagtctttgggttctggggggagtatggtcgcaaggctgaaacttaaaggaattgacggaagggcaccactaggagtggagcctgcggctaatttgactcaacacggggaaactcaccaggtccagacacaataaggattgacagattgagagctctttcttgattttgtgggtggtggtgcatggccgtttctcagttggtggagtgatttgtctgcttaattgcgataacgaacgagaccttaacctactaaatagtggtgctagcatttgctggttatccacttcttagagggactatcggtttcaagccgatggaagtttgaggcaataacaggtctgtgatgcccttagaacgttctgggccgcacgcgcgctacactgacggagccagcgagtctaaccttggccgagaggtcttggtaatcttgtgaaactccgtcgtgctggggatagagcattgtaattattgctcttcaacgaggaattcctagtaagcgcaagtcatcagcttgcgttgattacgtccctgccctttgtacacaccgcccgtcgctagtaccgattgaatggcttagtgaggcctcaggatctgcttagagaagggggcaactccatctcagagcggagaatttggacaaacttggtcatttagaggaactaaaagtcgtaacaaggtttccgtaggtgaacctgcggaaggatcatta

SEQ ID NO: 9 - DNA, 3911 bp, Yeast 25S Ribosomal RNA
aattccgtgatgggcctttaggttttaccaactgcggctaatctttttttatactgagcgtattggaacgttatcgataagaagagagcgtctaggcgaacaatgttcttaaagtttgacctcaaatcaggtaggagtacccgctgaacttaagcatatcaataagcggaggaaaagaaaccaaccggattgccttagtaacggcgagtgaagcggcaaaagctcaaatttgaaatctggtaccttcggtgcccgagttgtaatttggagagggcaactttggggccgttccttgtctatgttccttggaacaggacgtcatagagggtgagcatcccgtgtggcgaggagtgcggttctttgtaaagtgccttcgaagagtcgagttgtttgggaatgcagctctaagtgggtggtaaattccatctaaagctaaatattggcgagagaccgatagcgaacaagtacagtgatggaaagatgaaaagaactttgaaaagagagtgaaaaagtacgtgaaattgttgaaagggaagggcatttgatcagacatggtgttttgtgccctctgctccttgtgggtaggggaatctcgcatttcactgggccagcatcagttttggtggcaggataaatccataggaatgtagcttgcctcggtaagtattatagcctgtgggaatactgccagctgggactgaggactgcgacgtaagtcaaggatgctggcataatggttatatgccgcccgtcttgaaacacggaccaaggagtctaacgtctatgcgagtgtttgggtgtaaaacccatacgcgtaatgaaagtgaacgtaggttggggcctcgcaagaggtgcacaatcgaccgatcctgatgtcttcggatggatttgagtaagagcatagctgttgggacccgaaagatggtgaactatgcctgaatagggtgaagccagaggaaactctggtggaggctcgtagcggttctgacgtgcaaatcgatcgtcgaatttgggtataggggcgaaagactaatcgaaccatctagtagctggttcctgccgaagtttccctcaggatagcagaagctcgtatcagttttatgaggtaaagcgaatgattagaggttccggggtcgaaatgaccttgacctattctcaaactttaaatatgtaagaagtccttgttacttaattgaacgtggacatttgaatgaagagcttttagtgggccatttttggtaagcagaactggcgatgcgggatgaaccgaacgtagagttaaggtgccggaatacacgctcatcagacaccacaaaaggtgttagttcatctagacagccggacggtggccatggaagtcggaatccgctaaggagtgtgtaacaactcaccggccgaatgaactagccctgaaaatggatggcgctcaagcgtgttacctatactctaccgtcagggttgatatgatgccctgacgagtaggcaggcgtggaggtcagtgacgaagcctagaccgtaaggtcgggtcgaacggcctctagtgcagatcttggtggtagtagcaaatattcaaatgagaactttgaagactgaagtggggaaaggttccacgtcaacagcagttggacgtgggttagtcgatcctaagagatggggaagctccgtttcaaaggcctgattttatgcaggccaccatcgaaagggaatccggtaagattccggaacttggatatggattcttcacggtaacgtaactgaatgtggagacgtcggcgcgagccctgggaggagttatcttttcttcttaacagcttatcaccccggaattggtttatccggagatggggtcttatggctggaagaggccagcacctttgctggctccggtgcgcttgtgacggcccgtgaaaatccacaggaaggaatagttttcatgctaggtcgtactgataaccgcagcaggtctccaaggtgaacagcctctagttgatagaataatgtagataagggaagtcggcaaaatagatccgtaacttcgggataaggattggctctaagggtcgggtagtgagggccttggtcagacgcagcgggcgtgcttgtggactgcttggtggggcttgctctgctaggcggactacttgcgtgccttgttgtagacggccttggtaggtctcttgtagaccgtcgcttgctacaattaacagatcaacttagaactggtacggacaaggggaatctgactgtctaattaaaacatagcattgcgatggtcagaaagtgatgttgacgcaatgtgatttctgcccagtgctctgaatgtcaaagtgaagaaattcaaccaagcgcgagtaaacggcgggagtaactatgactctcttaaggtagccaaatgcctcgtcatctaattagtgacgcgcatgaatggattaacgagattcccactgtccctatctactatctagcgaaaccacagccaagggaacgggcttggcagaatcagcggggaaagaagaccctgttgagcttgactctagtttgacattgtgaagagacatagagggtgtagaataagtgggagcttcggcgccagtgaaataccactacctttatagtttctttacttattcaatgaagcggagctggaattcattttccacgttctagcattcaaggtcccattcggggctgatccgggttgaagacattgtcaggtggggagtttggctggggcggcacatctgttaaacgataacgcagatgtcctaaggggggctcatggagaacagaaatctccagtagaacaaaagggtaaagccccttagtttgatttcagtgtgaatacaaaccattgaaagtgtggcctatcgatcctttagtccctcggaatttgaggctagaggtgccagaaaagttaccacagggataactggcttgtggcagtcaagcgttcatagcgacattgctttttgattcttcgatgtcggctcttcctatcataccgaagcagaattcggtaagcgttggattgttcacccactaatagggaacatgagctgggtttagaccgtcgtgagacaggttagttttaccctactgatgaatgttaccagcaatagtaattgaacttagtacgagaggaacagttcattcggataattggtttttgcggctgtctgatcaggcattgccgcgaagcaccatccgctggattatggctgaacgcctctaagtcagaatccatgctagaacgcggtgatttctttgctccacacaatatagatggatacgaataaggcgtccttgtggcgtcgctgaaccatagcaggctagcaacggtgcacttggcggaaaggccttgggtgcttgctggcgaattgcaatgtcattttgcgtggggataaatcatttgtatacgacttagatgtacaacggggtattgtaagcggtagagtagccttgttgttacgatctgctgagattaagcctttgttgtctgatttgttttttatttctttctaagtgggtactggcaggagccggggcctagtttagagagaagtagactcaacaagtctctataaattttatttgtcttaagaattctatgatccgggtaaaaacatgtattgtatatatctattataatatacgatgaggatgatagtgtgtaagagtgtaccatttactaatgtatgtaagttactatttactatttggtctttttattttttattttttttttttttttcgttgcaaagatgggttgaaagagaagggctttcacaaagcttcccgagcgtgaaaggatttgcccggacagtttgcttcatggagcagttttttccgcaccatcagagcggcaaacatgagtgcttgtataagtttagagaattgagaaaagctcattt

SEQ ID NO: 10 - DNA, 16569 bp, Human mitochondrial DNA
gatcacaggtctatcaccctattaaccactcacgggagctctccatgcatttggtattttcgtctggggggtatgcacgcgatagcattgcgagacgctggagccggagcaccctatgtcgcagtatctgtctttgattcctgcctcatcctattatttatcgcacctacgttcaatattacaggcgaacatacttactaaagtgtgttaattaattaatgcttgtaggacataataataacaattgaatgtctgcacagccactttccacacagacatcataacaaaaaatttccaccaaaccccccctcccccgcttctggccacagcacttaaacacatctctgccaaaccccaaaaacaaagaaccctaacaccagcctaaccagatttcaaattttatcttttggcggtatgcacttttaacagtcaccccccaactaacacattattttcccctcccactcccatactactaatctcatcaatacaacccccgcccatcctacccagcacacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaagggacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactaagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctaccttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccagacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgaaccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaataacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaattaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggtcataaagcctaaatagcccacacgttccccttaaataagacatcacgatg

SEQ ID NO: 11 - DNA, 17 bp, B19 Virus
tggtctgggatgaaggt

SEQ ID NO: 12 - DNA, 23 bp, B19 virus
ccattttaggcgggcaacccacc

SEQ ID NO: 13 - DNA, 21 bp, B19 virus
tggaagtgtagctgtgcctgg

SEQ ID NO: 14 - DNA, 23 bp, B19 virus
agaatcatttgtcggaagctcag

SEQ ID NO: 15 - DNA, 20 bp, B19 virus
acaagcctgggcaagttagc

SEQ ID NO: 16 - DNA, 21 bp, B19 virus
acaatgccagtggaaaggagg

SEQ ID NO: 17 - DNA, 23 bp, B19 virus
ctttaacacatgaagaccatgca

SEQ ID NO: 18 - DNA, 28 bp, B19 virus
cctctcaaaacactagaatatccttacg

SEQ ID NO: 19 - DNA, 27 bp, B19 virus
cagccataccaccactgggacacagat

SEQ ID NO: 20 - DNA, 23 bp, B19 virus
aatgccatttctcatggtcagac

SEQ ID NO: 21 - DNA, 20 bp, B19 virus
gcaaaacaacaccacaggca

SEQ ID NO: 22 - DNA, 22 bp, Hepatitis B virus
caacctccaatcactcaccaac

SEQ ID NO: 23 - DNA, 24 bp, Hepatitis B virus
cctccaatttgtcctggttatcgc

SEQ ID NO: 24 - DNA, 23 bp, Hepatitis B virus
gtgtctgcggcgttttatcatat

SEQ ID NO: 25 - DNA, 23 bp, Hepatitis B virus
tgtttggctttcagctatatgga

SEQ ID NO: 26 - DNA, 22 bp, Hepatitis B virus
aattgtgggtcttttgggcttt

SEQ ID NO: 27 - DNA, 18 bp, Hepatitis B virus
ttctccgtctgccgttcc

SEQ ID NO: 28 - DNA, 21 bp, Hepatitis B virus
accagcaccatgcaacttttt

SEQ ID NO: 29 - DNA, 25 bp, Hepatitis B virus
tttttcacctctgcctaatcatctc

SEQ ID NO: 30 - DNA, 16 bp, Hepatitis B virus
tcccactgttcaagcc

SEQ ID NO: 31 - DNA, 21 bp, Hepatitis B virus
acctcaccataccgcactcag

SEQ ID NO: 32 - DNA, 25 bp, Hepatitis B virus
gggaattgatgactctagctacctg

SEQ ID NO: 33 - DNA, 22 bp, Hepatitis B virus
atcctgaatggcaaactccttc

SEQ ID NO: 34 - DNA, 20 bp, Hepatitis B virus
gagaaaccacacgtagcgca

SEQ ID NO: 35 - DNA, 16 bp, Hepatitis B virus
cacccctccacacggc

SEQ ID NO: 36 - DNA, 27 bp, Hepatitis B virus
atctctccacctctaagagacagtcat

SEQ ID NO: 37 - DNA, 22 bp, Porcine parvovirus
cctcacaaaacggcaagtactg

SEQ ID NO: 38 - DNA, 36 bp, Porcine parvovirus
acctaagtccaagtgactgctactggttcatacagc

SEQ ID NO: 39 - DNA, 29 bp, Porcine parvovirus
gttaataatgcaatgcaaagtacctctaa

SEQ ID NO: 40 - DNA, 25 bp, Porcine parvovirus
aaagacaactgaaagagagcatgga

SEQ ID NO: 41 - DNA, 30 bp, Porcine parvovirus
tctcagctcagattctggcttcatgacaaa

SEQ ID NO: 42 - DNA, 22 bp, Porcine parvovirus
caattctatttcatgggccagc

SEQ ID NO: 43 - DNA, 17 bp, Porcine parvovirus
cgtggagcgagccaaca

SEQ ID NO: 44 - DNA, 28 bp, Porcine parvovirus
ctgcacttaactccaacaccgccagatt

SEQ ID NO: 45 - DNA, 24 bp, Porcine parvovirus
gcaatacggacaccaagtccaact

SEQ ID NO: 46 - DNA, 23 bp, Porcine parvovirus
gaggtaagaagatcgccgagaaa

SEQ ID NO: 47 - DNA, 28 bp, Porcine parvovirus
aacctcaccaccaaccaaaatatataat

SEQ ID NO: 48 - DNA, 29 bp, Porcine parvovirus
actactaactgaacctaccacagaaggag

SEQ ID NO: 49 - DNA, 26 bp, Porcine parvovirus
cttttaccttcagatccaataggagg

SEQ ID NO: 50 - DNA, 18 bp, Sindbis virus
gcgtgcggaccctgtact

SEQ ID NO: 51 - DNA, 25 bp, Sindbis virus
attggcttcgacaccacccagttca

SEQ ID NO: 52 - DNA, 20 bp, Sindbis virus
ttctcggctatggcaggttc

SEQ ID NO: 53 - DNA, 25 bp, Sindbis virus
gtttatttctccgtaggatcgacac

SEQ ID NO: 54 - DNA, 20 bp, Sindbis virus
aaaactgctgcaggtctcgg

SEQ ID NO: 55 - DNA, 22 bp, Sindbis virus
gaaatcgatattacaggggcca

SEQ ID NO: 56 - DNA, 21 bp, Sindbis virus
gcattaagtttttcggcatgg

SEQ ID NO: 57 - DNA, 18 bp, Sindbis virus
cgattggcatagccggtg

SEQ ID NO: 58 - DNA, 21 bp, West Nile virus
tcagcgatctctccaccaaag

SEQ ID NO: 59 - DNA, 22 bp, West Nile virus
tgcccgaccatgggggaagccc

SEQ ID NO: 60 - DNA, 21 bp, West Nile virus
caatgacaaacgtgctgaccc

SEQ ID NO: 61 - DNA, 20 bp, West Nile virus
gctagtcctggtgtttgggg

SEQ ID NO: 62 - DNA, 24 bp, Escherichia coli 16S Ribosomal RNA
agagtttgatcatggctcagattg

SEQ ID NO: 63 - DNA, 24 bp, Escherichia coli 16S Ribosomal RNA
ctggcggcaggcctaacacatgca

SEQ ID NO: 64 - DNA, 21 bp, Escherichia coli 16S Ribosomal RNA
aataccgcataacgtcgcaag

SEQ ID NO: 65 - DNA, 20 bp, Escherichia coli 16S Ribosomal RNA
gatgcaacgcgaagaacctt

SEQ ID NO: 66 - DNA, 23 bp, Escherichia coli 16S Ribosomal RNA
gactggggtgaagtcgtaacaag

SEQ ID NO: 67 - DNA, 24 bp, Escherichia coli 16S Ribosomal RNA
gtaacaaggtaaccgtaggggaac

SEQ ID NO: 68 - DNA, 19 bp, Escherichia coli 16S Ribosomal RNA
gcggttggatcacctcctt

SEQ ID NO: 69 - DNA, 22 bp, Escherichia coli 23S Ribosomal RNA
ccgatagtgaaccagtaccgtg

SEQ ID NO: 70 - DNA, 25 bp, Escherichia coli 23S Ribosomal RNA
atgttgaaaaattagcggatgactt

SEQ ID NO: 71 - DNA, 18 bp, Escherichia coli 23S Ribosomal RNA
gcactgtttcggcaaggg

SEQ ID NO: 72 - DNA, 18 bp, Escherichia coli 23S Ribosomal RNA
gccggaagaccaagggtt

SEQ ID NO: 73 - DNA, 24 bp, Escherichia coli 23S Ribosomal RNA
ggccgtaactataacggtcctaag

SEQ ID NO: 74 - DNA, 26 bp, Escherichia coli 23S Ribosomal RNA
gataagtgctgaaagcatctaagcac

SEQ ID NO: 75 - DNA, 25 bp, Yeast (S. cerevisiae)
ctgccagtagtcatatgcttgtctc

SEQ ID NO: 76 - DNA, 36 bp, Yeast (S. cerevisiae)
tacagtgaaactgcgaatggctcattaaatcagtta

SEQ ID NO: 77 - DNA, 27 bp, Yeast (S. cerevisiae)
taatacatgcttaaaatctcgaccctt

SEQ ID NO: 78 - DNA, 24 bp, Yeast (S. cerevisiae)
gtcttcggactctttgatgattca

SEQ ID NO: 79 - DNA, 17 bp, Yeast (S. cerevisiae)
gcagccgcggtaattcc

SEQ ID NO: 80 - DNA, 24 bp, Yeast (S. cerevisiae)
gctgaaacttaaaggaattgacgg

SEQ ID NO: 81 - DNA, 23 bp, Yeast (S. cerevisiae)
tggaagtttgaggcaataacagg

SEQ ID NO: 82 - DNA, 19 bp, Yeast (S. cerevisiae)
tgaacctgcggaaggatca

SEQ ID NO: 83 - DNA, 24 bp, Yeast 25S Ribosomal RNA
aagcatatcaataagcggaggaaa

SEQ ID NO: 84 - DNA, 20 bp, Yeast 25S Ribosomal RNA
ctctggtggaggctcgtagc

SEQ ID NO: 85 - DNA, 18 bp, Yeast 25S Ribosomal RNA
aatggatggcgctcaagc

SEQ ID NO: 86 - DNA, 25 bp, Yeast 25S Ribosomal RNA
tgaaaatccacaggaaggaatagtt

SEQ ID NO: 87 - DNA, 21 bp, Yeast 25S Ribosomal RNA
ctaagggtcgggtagtgaggg

SEQ ID NO: 88 - DNA, 20 bp, Yeast 25S Ribosomal RNA
agaaattcaaccaagcgcga

SEQ ID NO: 89 - DNA, 19 bp, Yeast 25S Ribosomal RNA
atgtcattttgcgtgggga

SEQ ID NO: 90 - DNA, 23 bp, Human mitochondrial DNA
gttcaccctctaaatcaccacga

SEQ ID NO: 91 - DNA, 23 bp, Human mitochondrial DNA
caagcacgcagcaatgcagctca

SEQ ID NO: 92 - DNA, 26 bp, Human mitochondrial DNA
ggaaacagcagtgattaacctttagc

SEQ ID NO: 93 - DNA, 28 bp, Human mitochondrial DNA
gactacgaaagtggctttaacatatctg

SEQ ID NO: 94 - DNA, 24 bp, Human mitochondrial DNA
tagagtgcttagttgaacagggcc

SEQ ID NO: 95 - DNA, 24 bp, Human mitochondrial DNA
taggcgatagaaattgaaacctgg

SEQ ID NO: 96 - DNA, 21 bp, Human mitochondrial DNA
tttgttaagatggcagagccc

SEQ ID NO: 97 - DNA, 20 bp, Human mitochondrial DNA
agaatcgaacccatccctga

SEQ ID NO: 98 - DNA, 19 bp, Human mitochondrial DNA
tttcaccgtaggtggcctg

SEQ ID NO: 99 - DNA, 20 bp, Human mitochondrial DNA
aatcgctgtcgccttaatcc